(19) United States
(12) Patent Application Publication
(10) Pub. No.: US 2017/0112831 A1
(43) Pub. Date: Apr. 27, 2017

(54) USE OF CCR1 ANTAGONISTS AS A TREATMENT FOR TUMORS OF THE CENTRAL NERVOUS SYSTEM

(71) Applicants: James Merritt, West Windsor, NJ (US); Sal Coniglio, Fanwood, NJ (US)

(72) Inventors: James Merritt, West Windsor, NJ (US); Sal Coniglio, Fanwood, NJ (US)

(21) Appl. No.: 15/299,701

(22) Filed: Oct. 21, 2016

Related U.S. Application Data
(60) Provisional application No. 62/245,551, filed on Oct. 23, 2015.

(51) Int. Cl. A61K 31/498; A61K 31/40; A61K 31/4545; A61K 31/443; A61K 31/453; A61K 31/451; A61K 31/495; A61K 31/437; A61K 31/4439; A61K 31/55; A61K 31/4196; A61K 31/496

(52) U.S. Cl. CPC A61K 31/498; A61K 31/4196; A61K 31/40; A61K 31/4545; A61K 31/443; A61K 31/496; A61K 31/451; A61K 31/495; A61K 31/437; A61K 31/4439; A61K 31/55; A61K 31/453

(57) ABSTRACT

The present disclosure relates to Chemotactic Cytokine Receptor 1 (CCR1) antagonists and their use in the treatment of tumors of the central nervous system. More specifically, to the treatment of Astrocytic tumors.

### CROSS-REFERENCE TO RELATED APPLICATIONS

This application claims the benefit of priority to U.S. Provisional Application No. 62/245,551, filed Oct. 23, 2015, the entirety of which is incorporated herein by reference.

### TECHNICAL FIELD

This disclosure relates to Chemotactic Cytokine Receptor 1 (CCR1) antagonists and their use in the treatment of tumors of the central nervous system. More specifically, the present disclosure relates to the treatment of Astrocytic tumors.

### BACKGROUND

Chemotactic Cytokine Receptor 1 (CCR1) belongs to a large family (>20) of chemotactic cytokine (chemokine) receptors that interact with specific chemokines (>50) to mediate leukocyte trafficking, granule exocytosis, gene transcription, mitogenic effects and apoptosis. Chemokines are best known for their ability to mediate basal and inflammatory leukocyte trafficking. The ligands of this receptor include macrophage inflammatory protein 1 alpha (MIP-1 alpha/CCL3), regulated on activation normal T expressed and secreted protein (RANTES/CCL5), monocyte chemoattractant protein 3 (MCP-3/CCL7), and myeloid progenitor inhibitory factor-1 (MPIF-1). Chemokines and their receptors mediate signal transduction and are critical for the recruitment of effector immune cells to the site of inflammation.

Central nervous system tumors are relatively common in the United States, with more than 40,000 cases annually. Although more than half of these tumors are benign, they can cause substantial morbidity. Malignant primary brain tumors are the leading cause of death from solid tumors in children and the third leading cause of death from cancer in adolescents and adults aged 15 to 34 years. Common presenting symptoms include headache, seizures, and altered mental status. Whereas magnetic resonance imaging helps define the anatomic extent of tumor, biopsy is often required to confirm the diagnosis. Treatment depends on the histologic diagnosis. Benign tumors are usually curable with surgical resection or radiation therapy including stereotactic radiation; however, most patients with malignant brain tumors benefit from chemotherapy either at the time of initial diagnosis or at tumor recurrence. Metastases to the brain remain a frequent and morbid complication of solid tumors but are frequently controlled with surgery or radiation therapy. Unfortunately, the mortality rate from malignant brain tumors remains high, despite initial disease control.

Astrocytic tumours are the commonest type of cancer of the brain. They originate in a particular kind of glial cells, star-shaped brain cells in the cerebrum called astrocytes. This type of tumor does not usually spread outside the brain and spinal cord and it does not usually affect other organs. Astrocytomas can occur in most parts of the brain.

The most aggressive Astrocytic tumour is Glioblastoma, which is a World Health Organization (WHO) grade IV type (tumors which reproduce rapidly and are very aggressive malignant tumors). It is difficult to estimate incidence of specific types of Astrocytic tumours due to variable precision in the historic registration and coding of brain and CNS tumour types, but Astrocytomas make up over 80% of brain tumors and around 75% of Astrocytomas are highly aggressive Glioblastomas. Astrocytomas can be diagnosed at any age, but the WHO grade I types are more often found in children or young adults, while the WHO grade III and IV types are more prevalent in adults. The ability of glioblastoma tumor cells to infiltrate the surrounding brain parenchyma critically limits the effectiveness of current treatments.

### SUMMARY

The invention described herein is based at least in part on the finding that the expression of CCR1 ligands are upregulated in glioblastoma and tumor associated macrophages/microglia (TAMs). The disclosure also provides evidence that small molecule CCR1 antagonists were found to inhibit the ability of microglia to stimulate invasion of the glioblastoma cell line (GL261) using in-vitro invasion chamber assays. The disclosure is thus useful for treating or diagnosing patients with brain cancer in general, glioblastoma in particular.

One embodiment disclosed herein includes a method for the treatment of tumors of the central nervous system comprising administering to the patient a therapeutically effective amount of a CCR1 antagonist, or a pharmaceutically acceptable salt thereof.

In some embodiments, the tumor may be selected from a group consisting of astrocytic tumors, oligodendroglial tumors, oligoastrocytic tumors, ependymal tumors, choroid plexus tumors, neuronal and mixed neuronal-glial tumors, pineal tumors, embryonal tumors, neuroblastic tumors, Glial tumors, tumors of cranial and paraspinal nerves, tumors of the meninges, tumors of the hematopoietic system, germ cell tumors, and tumors of the sellar region.

In some embodiments, the CCR1 antagonist may be selected from a compound disclosed in Bioorganic & Medicinal Chemistry Letters (2005), 15(23), 5160-5164; Bioorganic & Medicinal Chemistry Letters (2007), 17(11), 3109-3112; Bioorganic & Medicinal Chemistry Letters (2007), 17(12), 3367-3372; Bioorganic & Medicinal Chemistry Letters (2010), 20(18), 5477-5479; Bioorganic & Medicinal Chemistry Letters (2013), 23(5), 1228-31; Bioorganic & Medicinal Chemistry Letters (2013), 23(13), 3833-40; Bioorganic & Medicinal Chemistry Letters (2014), 24(1), 108-12; British Journal of Pharmacology (2014), 171(22), 5127-5138; Clinical Pharmacology & Therapeutics (2011), 89(5), 726-34; Current Topics in Medicinal Chemistry (2010), 10(13), 1268-1277; Expert Opinion on Investigational Drugs (2005), 14(7), 785-796; 1 Biol. Chem. (1998), 273(25), 15687-92; J. Biol. Chem. (2003), 278(42), 40473-80; Journal of Medicinal Chemistry (2009), 52(5), 1295-1301; Letters in Drug Design & Discovery (2006), 3(10), 689-694; Methods and Principles in Medicinal Chemistry (2011), 46(Chemokine Receptors as Drug Targets), 323-338; Molecular Diversity (2008), 12(1), 17-23; Respiratory Medicine (2010), 104(9), 1297-303; US20040092529; US20120270870; US20120270879; US20130203803; US20140057937; WO/2004/039376; WO/2004/039377; WO/2004/043965; WO/2005/079769; WO/2006/066948; WO/2006/133802; WO/2007/002293; WO/2007/022257; WO/2007/073432; WO/2008/011392; WO/2008/147822; WO/2009/134666; WO/2009/137338; WO/2010/036632; and WO/2012/087782, or a pharmaceutically acceptable salt thereof each of which is incorporated by reference in its entirety.

In some embodiments, the CCR1 antogonist may include those compounds disclosed in Table 1, or a pharmaceutically acceptable salt thereof. In some embodiments, the CCR1 antagonist may include those compounds disclosed in Table 2, or a pharmaceutically acceptable salt thereof. In some embodiments, the CCR1 antogonist may include those compounds disclosed in Table 3, or a pharmaceutically acceptable salt thereof.

In some embodiments, the methods described herein may include the administration of a therapeutically effective amount of additional active agents that may be selected from the group consisting of Temozolomide, Bevacizumab, Carmustine, Lomustine, Semustine, cisplatin, carboplatin, vincristine, cyclophosphamide, and a combination thereof. In some embodiments, the pharaceutical compositions described herein may include a therapeutically effective amount of additional active agents that may be selected from the group consisting of Temozolomide, Bevacizumab, Carmustine, Lomustine, Semustine, cisplatin, carboplatin, vincristine, cyclophosphamide, and a combination thereof.

In some embodiments, the methods described herein may include administering a CCR1 antagonist and/or an additional active agent via a route of administration that may may be selected from the group consisting of intracerebral administration, intracerebroventricularl administration, oral administration, subcutaneous administration, intravenous administration, intranasal administration, topical administration, transdermal administration, intraperitoneal administration, intramuscular administration, intrapulmonary administration, vaginal administration, rectal administration, otological administration, neuro-otological administration, intraocular administration, subconjuctival administration, administration via anterior eye chamber injection, intravitreal administration, intrathecal administration, intracystical administration, intrapleural administration, administration via wound irrigation, intrabuccal administration, intra-abdominal administration, intra-articular administration, intra-aural administration, intrabronchial administration, intracap sular administration, intrameningeal administration, admininstration via inhalation, administration via endotracheal or endobronchial instillation, administration via direct instillation into pulmonary cavities, intraspinal administration, intrasynovial administration, intrathoracical administration, administration via thoracostomy irrigation, epidural administration, intratympanical administration, intraci sternal administration, intravascular administration, intraventricular administration, intraosseous administration, administration via irrigation of infected bone, and administration via application as part of any admixture with a prosthetic device.

In some embodiments, any of the CCR1 antagonists described herein (e.g., compounds #1, #2, #5, and/or #7) may be used in a method of treatment of glioblastoma, or in a pharmaceutical composition for treatment of glioblastoma, as disclosed in International Published Patent Application No. W02015/031782 and U.S. Published Patent Application No. 2015/0065781, the entirety of which are incorporated herein by reference.

In some embodiments, any of the CCR1 antagonists described herein (e.g., compounds #1, #2, #5, and/or #7) may be used in a method of treatment of glioblastoma, or in a pharmaceutical composition for treatment of glioblastoma, as disclosed in European Patent Publication No. 2670405 and U.S. Published Patent Application No. 2013/0310407, the entirety of which are incorporated herein by reference.

It is to be understood that both the foregoing general description and the following detailed description are exemplary and explanatory only and are not restrictive of the disclosure, as claimed.

### BRIEF DESCRIPTION OF THE DRAWINGS

FIG. 1 is a bar graph showing the effect of four CCR1 antagonists on microglia stimulated glioma invasion. All four compounds showed a decrease in the number of invasive glioma cells with compound #1 showing a greater than 50% reduction.

FIG. 2 is a bar graph showing the effect a CCR1 antagonist (compound #2) on microglia stimulated invasion of U87 human glioblastoma cells using a 3D invasion culture assay.

FIG. 3 illustratesa study indicating that glioma-stimulation of JNK/SAPK activity in microglia is dependent on CCR1 signalling.

### DETAILED DESCRIPTION

The present disclosure relates to methods and pharmaceutical compositions for the treatment of tumors of the central nervous system. More particularly, the present disclosure relates to Chemotactic Cytokine Receptor 1 (CCR1) antagonists for use in the treatment of tumors of the central nervous system.

In a related aspect the disclosure provides a method for antagonizing CCR1 in a human subject suffering from a tumor of the central nervous system, comprising administering to the human subject a small molecule CCR1 antagonist such that CCR1 activity in the human subject is inhibited and treatment is achieved.

CCR1 ligands (including CCL3 and CCL5) are upregulated in glioblastoma and tumor associated macrophages/microglia (TAMs). Small molecule CCR1 antagonists were found to interfere with glioma cell interaction with the microenvironment, a necessary step in the progression towards full malignancy. CCR1 antagonists were tested and found to inhibit the ability of microglia to stimulate invasion of the glioblastoma cell line (GL261) using in-vitro invasion chamber assays. It has since been determined that GL261 express CCR1 and are responsive to the chemokine CCL3.

Some embodiments provided herein relate to a method for treating a disease or disorder including, but not limited to, tumors of the central nervous system.

Definitions

Unless defined otherwise, all technical and scientific terms used herein have the same meaning as is commonly understood by one of ordinary skill in the art to which this disclosure belongs. All patents, published patent applications, and other publications are incorporated by reference in their entirety. In the event that there is a plurality of definitions for a term herein, those in this section prevail unless stated otherwise.

The skilled artisan will recognize that some structures described herein may be resonance forms or tautomers of compounds that may be fairly represented by other chemical structures, even when kinetically, the artisan recognizes that such structures are only a very small portion of a sample of such compound(s). Such compounds are clearly contemplated within the scope of this disclosure, though such resonance forms or tautomers are not represented herein.

The compounds provided herein may encompass various stereochemical forms. The compounds also encompass diastereomers as well as optical isomers, e.g., mixtures of enantiomers including racemic mixtures, as well as individual enantiomers and diastereomers, which arise as a consequence of structural asymmetry in certain compounds. Separation of the individual isomers or selective synthesis of the individual isomers is accomplished by application of various methods which are well known to practitioners in the art. Unless otherwise indicated, when a disclosed compound is named or depicted by a structure without specifying the stereochemistry and has one or more chiral centers, it is understood to represent all possible stereoisomers of the compound.

The term “administration” or “administering” refers to a method of providing a dosage of a CCR1 antagonist or pharmaceutical composition to a vertebrate or invertebrate, including a mammal, a bird, a fish, a reptile, or an amphibian, where the method is, e.g., orally, subcutaneously, intravenously, intralymphatic, intranasally, topically, transdermally, intraperitoneally, intramuscularly, intrapulmonarilly, vaginally, rectally, ontologically, neuro-otologically, intraocularly, subconjuctivally, via anterior eye chamber injection, intravitreally, intraperitoneally, intrathecally, intracystically, intrapleurally, via wound irrigation, intrabuccally, intra-abdominally, intra-articularly, intra-aurally, intrabronchially, intracapsularly, intrameningeally, via inhalation, via endotracheal or endobronchial instillation, via direct instillation into pulmonary cavities, intraspinally, intrasynovially, intrathoracically, via thoracostomy irrigation, epidurally, intratympanically, intracisternally, intravascularly, intraventricularly, intraosseously, via irrigation of infected bone, or via application as part of any admixture with a prosthetic device. The method of administration can vary depending on various factors, e.g., the components of the pharmaceutical composition, the site of the disease, the disease involved, and the severity of the disease.

A “diagnostic” as used herein is a compound, method, system, or device that assists in the identification or characterization of a health or disease state. The diagnostic can be used in standard assays as is known in the art.

The term “mammal” is used in its usual biological sense. Thus, it specifically includes humans, cattle, horses, monkeys, dogs, cats, mice, rats, cows, sheep, pigs, goats, and non-human primates, but also includes many other species.

The term “pharmaceutically acceptable carrier”, “pharmaceutically acceptable diluent” or “pharmaceutically acceptable excipient” includes any and all solvents, co-solvents, complexing agents, dispersion media, coatings, isotonic and absorption delaying agents and the like which are not biologically or otherwise undesirable. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. In addition, various adjuvants such as are commonly used in the art may be included. These and other such compounds are described in the literature, e.g., in the Merck Index, Merck & Company, Rahway, NJ. Considerations for the inclusion of various components in pharmaceutical compositions are described, e.g., in Gilman et al. (Eds.) (2010); Goodman and Gilman's: The Pharmacological Basis of Therapeutics, 12th Ed., The McGraw-Hill Companies which is incorporated by reference in its entirety.

The term “pharmaceutically acceptable salt” refers to salts that retain the biological effectiveness and properties of the compounds provided herein and, which are not biologically or otherwise undesirable. In many cases, the compounds provided herein are capable of forming acid and/or base salts by virtue of the presence of amino and/or carboxyl groups or groups similar thereto. Many such salts are known in the art, for example, as described in WO 87/05297. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium, and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine.

“Solvate” refers to the compound formed by the interaction of a solvent and a CCR1 antagonist as provided herein or a salt thereof. Suitable solvates are pharmaceutically acceptable solvates including hydrates.

“Patient” as used herein, means a human or a non-human mammal, e.g., a dog, a cat, a mouse, a rat, a cow, a sheep, a pig, a goat, a non-human primate, or a bird, e.g., a chicken, as well as any other vertebrate or invertebrate. In some embodiments, the patient is a human.

A “therapeutically effective amount” or “pharmaceutically effective amount” of a CCR1 antagonist as provided herein is one which is sufficient to achieve the desired physiological effect and may vary according to the nature and severity of the disease condition, and the potency of the CCR1 antagonist. “Therapeutically effective amount” is also intended to include one or more of the CCR1 antagonist in combination with one or more other agents that are effective to treat the diseases and/or conditions described herein. The combination of compounds can be a synergistic combination. Synergy, as described, for example, by Chou and Talalay, Advances in Enzyme Regulation (1984), 22, 27-55, occurs when the effect of the compounds when administered in combination is greater than the additive effect of the compounds when administered alone as a single agent. In general, a synergistic effect is most clearly demonstrated at sub-optimal concentrations of the compounds. It will be appreciated that different concentrations may be employed for prophylaxis than for treatment of an active disease. This amount can further depend upon the patient's height, weight, sex, age and medical history.

A “therapeutic effect” as that term is used herein, encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof. In certain embodiments, therapeutic effect relieves, to some extent, one or more of the symptoms of the disease, and can include curing a disease. “Curing” means that the symptoms of a disease are eliminated. However, certain long-term or permanent effects of the disease may exist even after a cure is obtained (such as extensive tissue damage).

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, etc. which can readily be determined by one of ordinary skill in the art. The term also applies to a dose that will induce a particular response in target cells (e.g., cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

“Treat,” “treatment,” or “treating,” as used herein refers to administering a CCR1 antagonist or pharmaceutical composition as provided herein for therapeutic purposes. The term “therapeutic treatment” refers to administering treatment to a patient already suffering from a disease thus causing a therapeutically beneficial effect, such as ameliorating existing symptoms, ameliorating the underlying metabolic causes of symptoms, postponing or preventing the further development of a disorder, and/or reducing the severity of symptoms that will or are expected to develop.

“Drug-eluting” and/or controlled release as used herein refers to any and all mechanisms, e.g., diffusion, migration, permeation, and/or desorption by which the drug(s) incorporated in the drug-eluting material pass therefrom over time into the surrounding body tissue.

“Drug-eluting material” and/or controlled release material as used herein refers to any natural, synthetic or semi-synthetic material capable of acquiring and retaining a desired shape or configuration and into which one or more drugs can be incorporated and from which incorporated drug(s) are capable of eluting over time.

“Elutable drug” as used herein refers to any drug or combination of drugs having the ability to pass over time from the drug-eluting material in which it is incorporated into the surrounding areas of the body.

As used herein the term “CCR1 antagonist” refers to any compound natural or not that is able to inhibit the activation of CCR1 by CCL3 also known as MIP-1 alpha. Typically said antagonist can inhibit binding of CCL3 to CCR1. Antagonistic activities of CCR1 may be determined according to any method well known in the art. Typically the assays may consist in a ligand-binding assay (inhibition of 1251-MIP-la binding to membranes prepared from transfected CHO cells) and in a functional assay by measuring inhibition of MIP-1 a induced Ca2+ flux in transfected CHO cells loaded with fluo-4 AM. For example a commercially—available cell line (Chemicon HTS001C) that stably co-expresses CCR1 and Gal 6 may be used. Said cell may be then loaded with calcium dye, pre-incubated with a test compounds and challenged with CCL3. Intracellular calcium flux may then measured and compared to control (CCL3, but no compound). For example such a method is described in the International Patent Application Publications WO 2010/036632, WO 2009/134666, and WO 2009/137338, and U.S. Pat. Nos. 7,879,873, 8,008,327, and 8,293,917, which are incorporated by reference in their entirety. CCR1 antagonists according to the disclosure may for example be selected from the group consisting of small organic molecules, antibodies, aptamers and polypeptides. In a particular embodiment, the CCR1 antagonist according to the disclosure is a small organic molecule. The term “active agent,” as used herein, may include a CCR1 antagonist.

The term “comprising” as used herein is synonymous with “including,” “containing,” or “characterized by,” and is inclusive or open-ended and does not exclude additional, unrecited elements or method steps.

Compounds

The CCR1 antagonists and compositions described herein can be used in the treatment of tumors of the central nervous system.

Suitable CCR1 antagonists are well known in the art, and an assay for determining CCR1 inhibition is also readily available using assays disclosed in the below noted patents or applications. Representative compounds which are inhibitors of CCR1 may be found in: Bioorganic & Medicinal Chemistry Letters (2005), 15(23), 5160-5164; Bioorganic & Medicinal Chemistry Letters (2007), 17(11), 3109-3112; Bioorganic & Medicinal Chemistry Letters (2007), 17(12), 3367-3372; Bioorganic & Medicinal Chemistry Letters (2010), 20(18), 5477-5479; Bioorganic & Medicinal Chemistry Letters (2013), 23(5), 1228-31; Bioorganic & Medicinal Chemistry Letters (2013), 23(13), 3833-40; Bioorganic & Medicinal Chemistry Letters (2014), 24(1), 108-12; British Journal of Pharmacology (2014), 171(22), 5127-5138; Clinical Pharmacology & Therapeutics (2011), 89(5), 726-34; Current Topics in Medicinal Chemistry (2010), 10(13), 1268-1277; Expert Opinion on Investigational Drugs (2005), 14(7), 785-796; J. Biol. Chem. (1998), 273(25), 15687-92; J. Biol. Chem. (2003), 278(42), 40473-80; Journal of Medicinal Chemistry (2009), 52(5), 1295-1301; Letters in Drug Design & Discovery (2006), 3(10), 689-694; Methods and Principles in Medicinal Chemistry (2011), 46(Chemokine Receptors as Drug Targets), 323-338; Molecular Diversity (2008), 12(1), 17-23; Respiratory Medicine (2010), 104(9), 1297-303; US20040092529; US20120270870; US20120270879; US20130203803; US20140057937; WO/2004/039376; WO/2004/039377; WO/2004/043965; WO/2005/079769; WO/2006/066948; WO/2006/133802; WO/2007/002293; WO/2007/022257; WO/2007/073432; WO/2008/011392; WO/2008/147822; WO/2009/134666; WO/2009/137338; WO/2010/036632; WO/2012/087782; whose disclosures are all incorporated herein by reference in their entirety.

Illustrative CCR1 antagonists are shown in Table 1.

TABLE 1

| | |
|---|---|
| 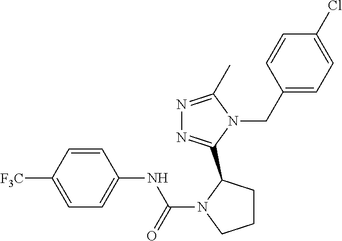 [SMILES: Cc1nnc([C@H]2CCCN2C(=O)Nc2ccc(C(F)(F)F)cc2)n1Cc1ccc(Cl)cc1] | 1 |
| 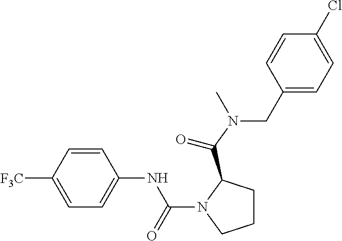 [SMILES: CN(Cc1ccc(Cl)cc1)C(=O)[C@H]1CCCN1C(=O)Nc1ccc(C(F)(F)F)cc1] | 2 |
| 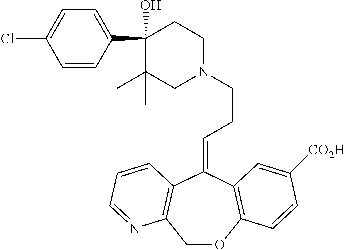 [SMILES: CC1(C)CN(CC/C=C2\c3cc(C(=O)O)ccc3OCc3ncccc32)CC[C@]1(O)c1ccc(Cl)cc1] | 3 |
| 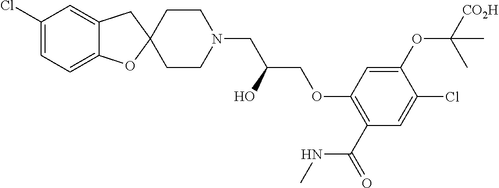 [SMILES: CNC(=O)c1cc(Cl)c(OC(C)(C)C(=O)O)cc1OC[C@@H](O)CN1CCC2(CC1)Cc1cc(Cl)ccc1O2] | 4 |
| 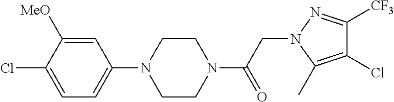 [SMILES: COc1cc(N2CCN(C(=O)Cn3nc(C(F)(F)F)c(Cl)c3C)CC2)ccc1Cl] | 5 |
| 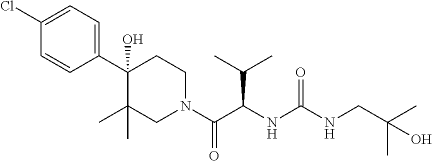 [SMILES: CC(C)[C@@H](NC(=O)NCC(C)(C)O)C(=O)N1CC[C@](O)(c2ccc(Cl)cc2)C(C)(C)C1] | 6 |
| 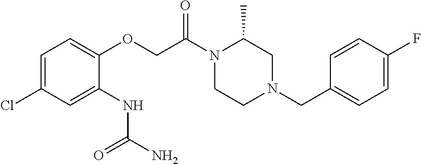 [SMILES: C[C@@H]1CN(Cc2ccc(F)cc2)CCN1C(=O)COc1ccc(Cl)cc1NC(N)=O] | 7 |
| 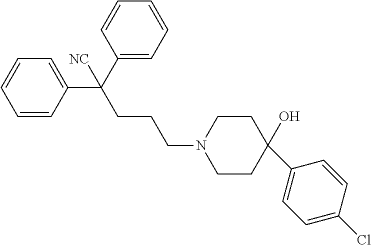 [SMILES: N#CC(CCCN1CCC(O)(c2ccc(Cl)cc2)CC1)(c1ccccc1)c1ccccc1] | 8 |
| 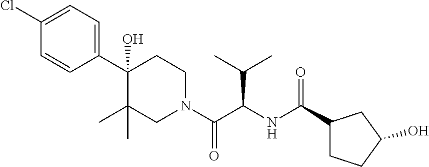 | 9 |
| 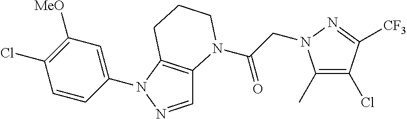 [SMILES: COc1cc(-n2ncc3c2CCCN3C(=O)Cn2nc(C(F)(F)F)c(Cl)c2C)ccc1Cl] | 10 |
| 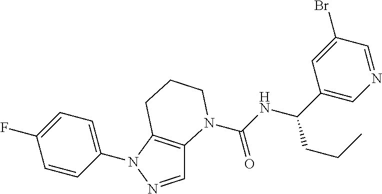 [SMILES: CCC[C@H](NC(=O)N1CCCc2c1cnn2-c1ccc(F)cc1)c1cncc(Br)c1] | 11 |
| 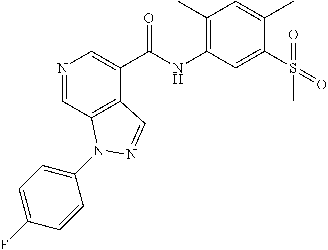 [SMILES: Cc1cc(C)c(S(C)(=O)=O)cc1NC(=O)c1cncc2c1cnn2-c1ccc(F)cc1] | 12 |
| 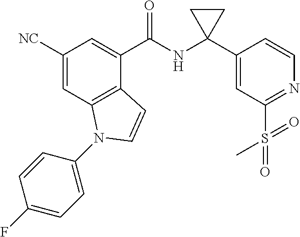 [SMILES: CS(=O)(=O)c1cc(C2(NC(=O)c3cc(C#N)cc4c3ccn4-c3ccc(F)cc3)CC2)ccn1] | 13 |
| 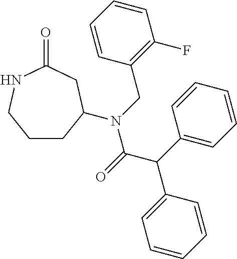 [SMILES: O=C1CC(N(Cc2ccccc2F)C(=O)C(c2ccccc2)c2ccccc2)CCCN1] | 14 |
| 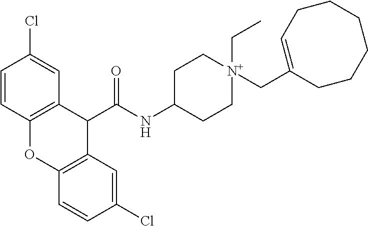 [SMILES: CC[N+]1(C/C2=C/CCCCCC2)CCC(NC(=O)C2c3cc(Cl)ccc3Oc3ccc(Cl)cc32)CC1] | 15 |
| 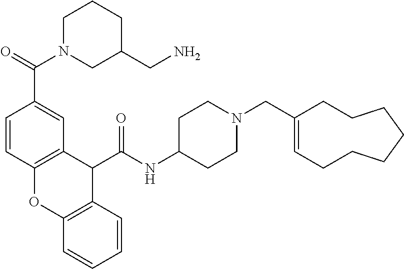 [SMILES: NCC1CCCN(C(=O)c2ccc3c(c2)C(C(=O)NC2CCN(C/C4=C/CCCCCCC4)CC2)c2ccccc2O3)C1] | 16 |
| 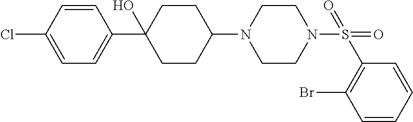 [SMILES: O=S(=O)(c1ccccc1Br)N1CCN(C2CCC(O)(c3ccc(Cl)cc3)CC2)CC1] | 17 |
| 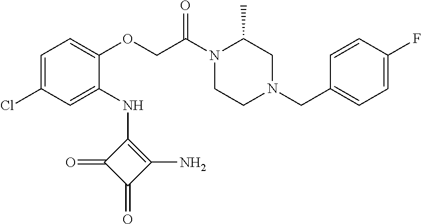 [SMILES: C[C@@H]1CN(Cc2ccc(F)cc2)CCN1C(=O)COc1ccc(Cl)cc1Nc1c(N)c(=O)c1=O] | 18 |
| 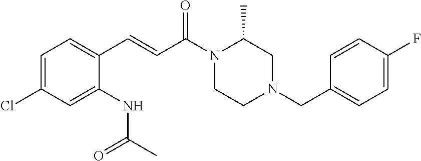 [SMILES: CC(=O)Nc1cc(Cl)ccc1/C=C/C(=O)N1CCN(Cc2ccc(F)cc2)C[C@H]1C] | 19 |
| 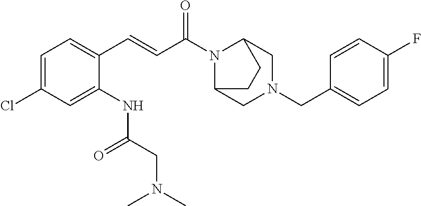 [SMILES: CN(C)CC(=O)Nc1cc(Cl)ccc1/C=C/C(=O)N1C2CCC1CN(Cc1ccc(F)cc1)C2] | 20 |
| 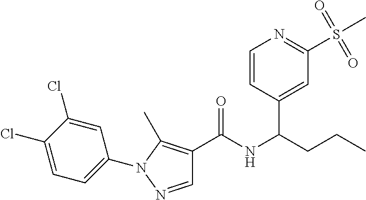 [SMILES: CCCC(NC(=O)c1cnn(-c2ccc(Cl)c(Cl)c2)c1C)c1ccnc(S(C)(=O)=O)c1] | 21 |
| 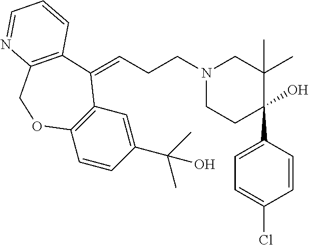 [SMILES: CC(C)(O)c1ccc2c(c1)/C(=C\CCN1CC[C@](O)(c3ccc(Cl)cc3)C(C)(C)C1)c1cccnc1CO2] | 22 |
| 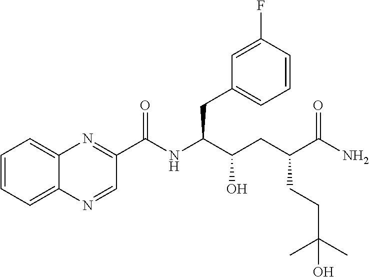 [SMILES: CC(C)(O)CC[C@H](C[C@H](O)[C@H](Cc1cccc(F)c1)NC(=O)c1cnc2ccccc2n1)C(N)=O] | 23 |
| 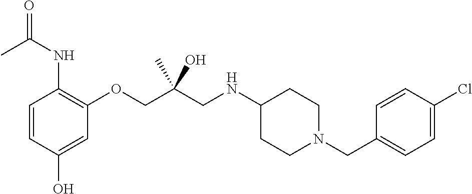 [SMILES: CC(=O)Nc1ccc(O)cc1OC[C@@](C)(O)CNC1CCN(Cc2ccc(Cl)cc2)CC1] | 24 |

In some embodiments, selected CCR1 antagonists of the invention include the compounds of Table 2.

TABLE 2

| | |
|---|---|
| 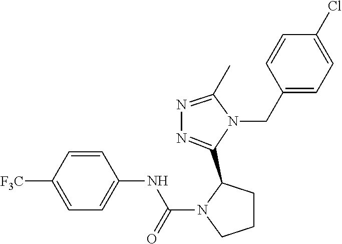 [SMILES: Cc1nnc([C@H]2CCCN2C(=O)Nc2ccc(C(F)(F)F)cc2)n1Cc1ccc(Cl)cc1] | 1 |
| 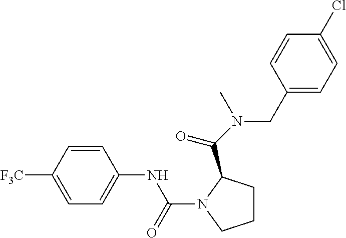 [SMILES: CN(Cc1ccc(Cl)cc1)C(=O)[C@H]1CCCN1C(=O)Nc1ccc(C(F)(F)F)cc1] | 2 |
| 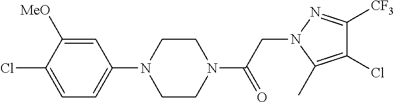 [SMILES: COc1cc(N2CCN(C(=O)Cn3nc(C(F)(F)F)c(Cl)c3C)CC2)ccc1Cl] | 5 |
| 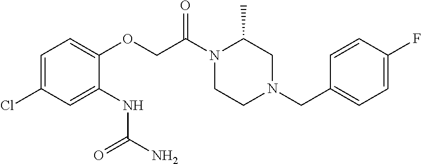 [SMILES: C[C@@H]1CN(Cc2ccc(F)cc2)CCN1C(=O)COc1ccc(Cl)cc1NC(N)=O] | 7 |

In some embodiments, selected CCR1 antagonists of the invention include the compounds of Table 3.

TABLE 3

| | |
|---|---|
| 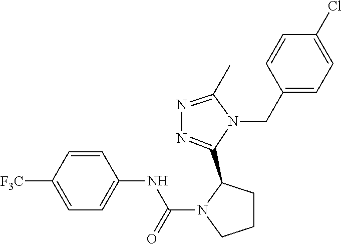 [SMILES: Cc1nnc([C@H]2CCCN2C(=O)Nc2ccc(C(F)(F)F)cc2)n1Cc1ccc(Cl)cc1] | 1 |
| 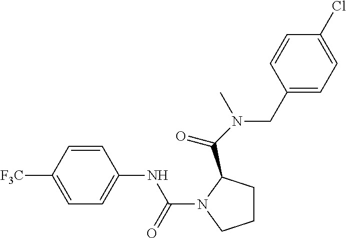 [SMILES: CN(Cc1ccc(Cl)cc1)C(=O)[C@H]1CCCN1C(=O)Nc1ccc(C(F)(F)F)cc1] | 2 |

### Administration and Pharmaceutical Compositions

Some embodiments include pharmaceutical compositions comprising: (a) a therapeutically effective amount of a CCR1 antagonist provided herein, or its corresponding enantiomer, diastereoisomer or tautomer, or pharmaceutically acceptable salt; and (b) a pharmaceutically acceptable carrier.

Non-limiting examples of tumors of the central nervous system which can be treated with a combination of a CCR1 antagonist and other known agents are astrocytic tumors, oligodendroglial tumors, oligoastrocytic tumors, ependymal tumors, choroid plexus tumors, neuronal and mixed neuronal-glial tumors, pineal tumors, embryonal tumors, neuroblastic tumors, Glial tumors, tumors of cranial and paraspinal nerves, tumors of the meninges, tumors of the hematopoietic system, germ cell tumors, and tumors of the sellar region.

In some embodiments, tumors of the central nervous system can be treated using a CCR1 antagonist or composition as described herein in combination with existing methods of treating cancers, for example by chemotherapy, irradiation, or surgery (e.g., oophorectomy). In some embodiments, a CCR1 antagonist or composition can be administered before, during, or after another anticancer agent or treatment.

The CCR1 antagonists provided herein may also be useful in combination (administered together or sequentially) with other known agents.

In some embodiments, tumors of the central nervous system can be treated with a combination of a CCR1 antagonist and an additional active agent, which may be one or more of the following drugs: Temozolomide (see, e.g., U.S. Pat. No. 5,939,098), Bevacizumab (see, e.g., U.S. Pat. No. 8,747,852), Carmustine (see, e.g., U.S. Published Application No. 2003/0082685), Lomustine (see, e.g., U.S. Published Application No. 2003/0082685), Semustine, cisplatin (see, e.g., U.S. Pat. No. 8,268,797), carboplatin (see, e.g., U.S. Pat. No. 7,008,936), vincristine (see, e.g., U.S. Pat. No. 6,271,245), or cyclophosphamide (see, e.g., U.S. Published Patent Application No. 2004/0014694).

Administration of the CCR1 antagonists disclosed herein or the pharmaceutically acceptable salts thereof can be via any of the accepted modes of administration, including, but not limited to, intracerebrally, intracerebroventricularly, orally, subcutaneously, intravenously, intranasally, topically, transdermally, intraperitoneally, intramuscularly, intrapulmonarilly, vaginally, rectally, otologically, neuro-otologically, intraocularly, subconjuctivally, via anterior eye chamber injection, intravitreally, intraperitoneally, intrathecally, intracystically, intrapleurally, via wound irrigation, intrabuccally, intra-abdominally, intra-articularly, intra-aurally, intrabronchially, intracapsularly, intrameningeally, via inhalation, via endotracheal or endobronchial instillation, via direct instillation into pulmonary cavities, intraspinally, intrasynovially, intrathoracically, via thoracostomy irrigation, epidurally, intratympanically, intracisternally, intravascularly, intraventricularly, intraosseously, via irrigation of infected bone, or via application as part of any admixture with a prosthetic devices. In some embodiments, the administration method includes oral or parenteral administration.

CCR1 antagonists provided herein intended for pharmaceutical use may be administered as crystalline or amorphous products. Pharmaceutically acceptable compositions may include solid, semi-solid, liquid, solutions, colloidal, liposomes, emulsions, suspensions, complexes, coacervates and aerosols. Dosage forms, such as, e.g., tablets, capsules, powders, liquids, suspensions, suppositories, aerosols, implants, controlled release or the like. They may be obtained, for example, as solid plugs, powders, or films by methods such as precipitation, crystallization, milling, grinding, supercritical fluid processing, coacervation, complex coacervation, encapsulation, emulsification, complexation, freeze drying, spray drying, or evaporative drying. Microwave or radio frequency drying may be used for this purpose. The compounds can also be administered in sustained or controlled release dosage forms, including depot injections, osmotic pumps, pills (tablets and or capsules), transdermal (including electrotransport) patches, biodegradable discs, implants and the like, for prolonged and/or timed, pulsed administration at a predetermined rate.

The CCR1 antagonists can be administered either alone or in combination with a conventional pharmaceutical carrier, excipient or the like. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium tri silicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a compound as described herein in the range of 0.005% to 100% with the balance made up from non-toxic carrier may be prepared. The contemplated compositions may contain 0.001%-100% of a CCR1 antagonist provided herein, in one embodiment 0.1-95%, in another embodiment 75-85%, in a further embodiment 20-80%. In still other embodiments the contemplated compositions may contain about 0.1-5%, 0.1-10%, 1-5%, 1-10%, 1-20%, 1-50%, 5-10%, 5-20%, 5-25%, 2-50%, 5-75%, 10-20%, 10-25%, 10-50%, 10-75%, 10-80%, 20-50%, 20-75%, 50-75%, or 50-85% of a CCR1 antagonist provided herein. In some embodiments the contemplated compositions may contain about 0.001%, 0.005%, 0.01%, 0.05%, 0.01%, 0.05%, 0.1%, 0.2%, 0.25%, 0.5%, 0.75%, 1%, 1.25%, 1.5%, 1.75%, 2%, 2.5%, 3%, 4%, 5%, 7.5%, 10%, 15%, 20%, 25%, 30%, 40%, 50%, 60%, 70%, 75%, 80%, 90%, 95%, or 99% of a CCR1 antagonist provided herein. Actual methods of preparing such dosage forms are known, or will be apparent, to those skilled in this art; for example, see Remington: The Science and Practice of Pharmacy, 22^{nd} Edition (Pharmaceutical Press, London, UK. 2012) which is incorporated by reference in its entirety.

In one embodiment, the compositions will take the form of a unit dosage form such as a pill or tablet and thus the composition may contain, along with a compound provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more CCR1 antagonists provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Liquid pharmaceutically administrable compositions can, for example, be prepared by dissolving, dispersing, etc. a CCR1 antagonist provided herein and optional pharmaceutical adjuvants in a carrier (e.g., water, saline, aqueous dextrose, glycerol, glycols, ethanol or the like) to form a solution, colloid, liposome, emulsion, complexes, coacervate or suspension. If desired, the pharmaceutical composition can also contain minor amounts of nontoxic auxiliary substances such as wetting agents, emulsifying agents, co-solvents, solubilizing agents, pH buffering agents and the like (e.g., sodium acetate, sodium citrate, cyclodextrin derivatives, sorbitan monolaurate, triethanolamine acetate, triethanolamine oleate, and the like).

In some embodiments, the compositions are provided in unit dosage forms suitable for single administration.

In some embodiments, the compositions are provided in unit dosage forms suitable for twice a day administration.

In some embodiments, the compositions are provided in unit dosage forms suitable for three times a day administration.

In some embodiments, the compositions are provided in unit dosage forms suitable for administration every other day, administration once a week, administration every other week, or administration once a month.

Injectables can be prepared in conventional forms, either as liquid solutions, colloid, liposomes, complexes, coacervate or suspensions, as emulsions, or in solid forms suitable for reconstitution in liquid prior to injection. The percentage of a compound provided herein contained in such parenteral compositions is highly dependent on the specific nature thereof, as well as the activity of the compound and the needs of the patient. However, percentages of active ingredient of 0.01% to 10% in solution are employable, and could be higher if the composition is a solid or suspension, which could be subsequently diluted to the above percentages.

In some embodiments, the composition will comprise about 0.1-10% of the active agent in solution.

In some embodiments, the composition will comprise about 0.1-5% of the active agent in solution.

In some embodiments, the composition will comprise about 0.1-4% of the active agent in solution.

In some embodiments, the composition will comprise about 0.15-3% of the active agent in solution.

In some embodiments, the composition will comprise about 0.2-2% of the active agent in solution.

In some embodiments, the compositions are provided in dosage forms suitable for continuous dosage by intravenous infusion over a period of about 1-96 hours.

In some embodiments, the compositions are provided in dosage forms suitable for continuous dosage by intravenous infusion over a period of about 1-72 hours.

In some embodiments, the compositions are provided in dosage forms suitable for continuous dosage by intravenous infusion over a period of about 1-48 hours.

In some embodiments, the compositions are provided in dosage forms suitable for continuous dosage by intravenous infusion over a period of about 1-24 hours.

In some embodiments, the compositions are provided in dosage forms suitable for continuous dosage by intravenous infusion over a period of about 1-12 hours.

In some embodiments, the compositions are provided in dosage forms suitable for continuous dosage by intravenous infusion over a period of about 1-6 hours.

In some embodiments, these compositions can be administered by intravenous infusion to humans at doses of about 5 mg/m² to about 300 mg/m².

In some embodiments, these compositions can be administered by intravenous infusion to humans at doses of about 5 mg/m² to about 200 mg/m².

In some embodiments, these compositions can be administered by intravenous infusion to humans at doses of about 5 mg/m² to about 100 mg/m².

In some embodiments, these compositions can be administered by intravenous infusion to humans at doses of about 10 mg/m² to about 50 mg/m².

In some embodiments, these compositions can be administered by intravenous infusion to humans at doses of about 50 mg/m² to about 200 mg/m².

In some embodiments, these compositions can be administered by intravenous infusion to humans at doses of about 75 mg/m² to about 175 mg/m².

In some embodiments, these compositions can be administered by intravenous infusion to humans at doses of about 100 mg/m² to about 150 mg/m².

It is to be noted that concentrations and dosage values may also vary depending on the specific compound and the severity of the condition to be alleviated. It is to be further understood that for any particular patient, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

In one embodiment, the compositions can be administered to the respiratory tract (including nasal and pulmonary) e.g., through a nebulizer, metered-dose inhalers, atomizer, mister, aerosol, dry powder inhaler, insufflator, liquid instillation or other suitable device or technique.

In some embodiments, aerosols intended for delivery to the nasal mucosa are provided for inhalation through the nose. For optimal delivery to the nasal cavities, inhaled particle sizes of about 5 to about 100 microns are useful, with particle sizes of about 10 to about 60 microns being preferred. For nasal delivery, a larger inhaled particle size may be desired to maximize impaction on the nasal mucosa and to minimize or prevent pulmonary deposition of the administered formulation. In some embodiments, aerosols intended for delivery to the lung are provided for inhalation through the nose or the mouth. For delivery to the lung, inhaled aerodynamic particle sizes of about less than 10 p.m are useful (e.g., about 1 to about 10 microns). Inhaled particles may be defined as liquid droplets containing dissolved drug, liquid droplets containing suspended drug particles (in cases where the drug is insoluble in the suspending medium), dry particles of pure drug substance, drug substance incorporated with excipients, liposomes, emulsions, colloidal systems, coacervates, aggregates of drug nanoparticles, or dry particles of a diluent which contain embedded drug nanoparticles.

In some embodiments, CCR1 antagonists disclosed herein intended for respiratory delivery (either systemic or local) can be administered as aqueous formulations, as non-aqueous solutions or suspensions, as suspensions or solutions in halogenated hydrocarbon propellants with or without alcohol, as a colloidal system, as emulsions, coacervates, or as dry powders. Aqueous formulations may be aerosolized by liquid nebulizers employing either hydraulic or ultrasonic atomization or by modified micropump systems (like the soft mist inhalers, the Aerodose® or the AERx® systems). Propellant-based systems may use suitable pressurized metered-dose inhalers (pMDIs). Dry powders may use dry powder inhaler devices (DPIs), which are capable of dispersing the drug substance effectively. A desired particle size and distribution may be obtained by choosing an appropriate device.

If desired, formulations of the disclosure can be incorporated into a gel formulation (e.g., U.S. Pat. Nos. 4,474,752 and 6,911,211, each incorporated by reference in its entirety).

Solid compositions can be provided in various different types of dosage forms, depending on the physicochemical properties of the CCR1 antagonist provided herein, the desired dissolution rate, cost considerations, and other criteria. In one of the embodiments, the solid composition is a single unit. This implies that one unit dose of the compound is comprised in a single, physically shaped solid form or article. In other words, the solid composition is coherent, which is in contrast to a multiple unit dosage form, in which the units are incoherent.

Examples of single units which may be used as dosage forms for the solid composition include tablets, such as compressed tablets, film-like units, foil-like units, wafers, lyophilized matrix units, and the like. In one embodiment, the solid composition is a highly porous lyophilized form. Such lyophilizates, sometimes also called wafers or lyophilized tablets, are particularly useful for their rapid disintegration, which also enables the rapid dissolution of the compound.

On the other hand, for some applications the solid composition may also be formed as a multiple unit dosage form as defined above. Examples of multiple units are powders, granules, microparticles, pellets, mini-tablets, beads, lyophilized powders, and the like. In one embodiment, the solid composition is a lyophilized powder. Such a dispersed lyophilized system comprises a multitude of powder particles, and due to the lyophilization process used in the formation of the powder, each particle has an irregular, porous microstructure through which the powder is capable of absorbing water very rapidly, resulting in quick dissolution. Effervescent compositions are also contemplated to aid the quick dispersion and absorption of the compound.

Another type of multiparticulate system which is also capable of achieving rapid drug dissolution is that of powders, granules, or pellets from water-soluble excipients which are coated with a compound provided herein so that the compound is located at the outer surface of the individual particles. In this type of system, the water-soluble low molecular weight excipient may be useful for preparing the cores of such coated particles, which can be subsequently coated with a coating composition comprising the compound and, for example, one or more additional excipients, such as a binder, a pore former, a saccharide, a sugar alcohol, a film-forming polymer, a plasticizer, or other excipients used in pharmaceutical coating compositions.

### Methods of Treatment

The CCR1 antagonists and their compositions provided herein can be used to treat tumors of the central nervous system.

More particularly, cancers that may be treated by the CCR1 antagonists, compositions and methods described herein include, but are not limited to, the following:

1) Astrocytic tumors, e.g., diffuse astrocytoma (fibrillary, protoplasmic, gemistocytic, mixed), anaplastic (malignant) astrocytoma, glioblastoma multiforme (giant cell glioblastoma and gliosarcoma), pilocytic astrocytoma (pilomyxoid astrocytoma), pleomorphic xanthoastrocytoma, subependymal giant cell astrocytoma, and gliomatosis cerebri.

2) Oligodendroglial tumors, e.g., oligodendroglioma and anaplastic oligodendroglioma.

3) Oligoastrocytic tumors, e.g., oligoastrocytoma and anaplastic oligoastrocytoma.

4) Ependymal tumors, e.g., subependymoma, myxopapillary ependymoma, ependymoma, (cellular, papillary, clear cell, tanycytic), and anaplastic (malignant) ependymoma.

5) Choroid plexus tumors, e.g., choroid plexus papilloma, atypical choroid plexus papilloma, and choroid plexus carcinoma.

6) Neuronal and mixed neuronal-glial tumors, e.g., gangliocytoma, ganglioglioma, dysembryoplastic neuroepithelial tumor (DNET), dysplastic gangliocytoma of the cerebellum (Lhermitte-Duclos), desmoplastic infantile astrocytoma/ganglioglioma, central neurocytoma, anaplastic ganglioglioma, extraventricular neurocytoma, cerebellar liponeurocytoma, Papillary glioneuronal tumor, Rosette-forming glioneuronal tumor of the fourth ventricle, and paraganglioma of the filum terminale.

7) Pineal tumors, e.g., pineocytoma, pineoblastoma, papillary tumors of the pineal region, and pineal parenchymal tumor of intermediate differentiation.

8) Embryonal tumors, e.g., medulloblastoma (medulloblastoma with extensive nodularity, anaplastic medulloblastoma, desmoplastic, large cell, melanotic, medullomyoblastoma), medulloepithelioma, supratentorial primitive neuroectodermal tumors, and primitive neuroectodermal tumors (PNETs) such as neuroblastoma, ganglioneuroblastoma, ependymoblastoma, and atypical teratoid/rhabdoid tumor.

9) Neuroblastic tumors, e.g., olfactory (esthesioneuroblastoma), olfactory neuroepithelioma, and neuroblastomas of the adrenal gland and sympathetic nervous system.

10) Glial tumors, e.g., astroblastoma, chordoid glioma of the third ventricle, and angiocentric glioma.

11) Tumors of cranial and paraspinal nerves, e.g., schwannoma, neurofibroma Perineurioma, and malignant peripheral nerve sheath tumor.

12) Tumors of the meninges such as tumors of meningothelial cells, e.g., meningioma (atypical meningioma and anaplastic meningioma); mesenchymal tumors, e.g., lipoma, angiolipoma, hibernoma, liposarcoma, solitary fibrous tumor, fibrosarcoma, malignant fibrous histiocytoma, leiomyoma, leiomyosarcoma, rhabdomyoma, rhabdomyosarcoma, chondroma, chondrosarcoma, osteoma, osteosarcoma, osteochondroma, haemangioma, epithelioid hemangioendothelioma, haemangiopericytoma, anaplastic haemangiopericytoma, angiosarcoma, Kaposi Sarcoma, and Ewing Sarcoma; primary melanocytic lesions, e.g., diffuse melanocytosis, melanocytoma, malignant melanoma, meningeal melanomatosis; and hemangioblastomas.

13) Tumors of the hematopoietic system, e.g., malignant Lymphomas, plasmocytoma, and granulocytic sarcoma.

14) Germ cell tumors, e.g., germinoma, embryonal carcinoma, yolk sac tumor, choriocarcinoma, teratoma, and mixed germ cell tumors.

15) Tumors of the sellar region, e.g., craniopharyngioma, granular cell tumor, pituicytoma, and spindle cell oncocytoma of the adenohypophysis.

Some embodiments of the present disclosure include methods for the treatment of tumors of the central nervous system comprising administering to the patient a therapeutically effective amount of a CCR1 antagonist, or a pharmaceutically acceptable salt thereof.

In some embodiments, the tumor is selected from a group consisting of astrocytic tumors, oligodendroglial tumors, oligoastrocytic tumors, ependymal tumors, choroid plexus tumors, neuronal and mixed neuronal-glial tumors, pineal tumors, embryonal tumors, neuroblastic tumors, Glial tumors, tumors of cranial and paraspinal nerves, tumors of the meninges, tumors of the hematopoietic system, germ cell tumors, and tumors of the sellar region.

In some embodiments, the tumor is an astrocytic tumor.

In some embodiments, the astrocytic tumor is glioblastoma multiforme.

Some embodiments of the present disclosure include methods for treating a disease characterized by tumor-associated microglia and glioblastoma invasion.

Some embodiments include methods for treating a disease characterized by tumor-associated macrophage and tumor cell invasion.

Some embodiments include methods for treating a disease characterized by glioma cell invasion.

Some embodiments include methods for treating a disease characterized by tumor cell invasion.

Some embodiments include methods for treating a disease mediated or sustained through the activity of CCR1.

Some embodiments include methods for treating a cancer mediated or sustained through the activity of CCR1.

Some embodiments include methods for blocking the binding of CCR1 to its ligands.

In some embodiments, CCR1 ligands include CCL2 (MCP-1), CCL3 (MIP-1α), CCL4 (MIP-1β), CCL5 (RANTES), CCL6 (MRP-1), CCL7 (MCP-3), CCL8 (MCP-2), CCL13 (MCP-4), CCL14 (HCC-1), CCL15 (Lkn-1), CCL16 (Monotactin-1), and CCL23 (MPIF-1).

### Evaluation of Biological Activity

The CCR1 antagonist activity of the compounds described herein can be assessed using suitable assays, such as receptor binding assays or chemotaxis assays. For example small molecule antagonists of MIP-1 binding have been identified utilizing THP-1 cell membranes. Specifically, a high through-put receptor binding assay, which monitors ¹²⁵I-MIP-1α binding to THP-1 cell membranes, has been used to identify small molecule antagonists which block binding of MIP-1α. Compounds of the present disclosure can also be identified by virtue of their ability to inhibit the activation steps triggered by binding of a chemokine (e.g., CCL2 (MCP-1), CCL3 (MIP-1α), CCL4 (MIP-1β), CCL5 (RANTES), CCL7 (MCP-3), CCL8 (MCP-2), CCL13 (MCP-4), CCL14 (HCC-1), CCL15 (Lkn-1), CCL23 (MPIF-1)) to its receptor (CCR-1), such as chemotaxis, integrin activation and granule mediator release. They can also be identified by virtue of their ability to block chemokine (e.g., CCL2 (MCP-1) CCL3 (MIP-1α), CCL4 (MIP-1β), CCL5 (RANTES), CCL7 (MCP-3), CCL8 (MCP-2), CCL13 (MCP-4), CCL14 (HCC-1), CCL15 (Lkn-1), CCL23 (MPIF-1)) induced chemotaxis of, for example, HL-60 cells, T-cells, peripheral blood mononuclear cells or eosinophils.

To determine the role of CCR1 on invasion, experimentation was first performed to determine how microglia, the resident macrophages of the brain, stimulate glioblastoma cell invasion. This was done by examining the ability of normal microglia from C57B1/6J mice to stimulate GL261 glioblastoma cell invasion in vitro. It was found that microglia stimulate the invasion of GL261 glioblastoma cells by approximately eightfold in an in vitro invasion assay. It was then determined that both GL261 cells and microglia cells expressed CCR1 and both are responsive to the chemokine CCL3. Blockade of CCR1 signaling in vivo reduced the number of tumor-associated microglia and glioblastoma invasion.

The biological activity of the compounds described herein can be tested according to any method well known in the art. Example methods can be found in Methods (2005), 37(2), 208-215; Mutation Research/Reviews in Mutation Research (2013), 752(1), 10-24 and Pharmaceutics (2011), 3(1), 107-124, each incorporated by reference in its entirety. Some examples of in vitro cell migration and invasion assays are outlined below.

In one example, microglia and/or glioblastoma cell migration and invasion can be evaluated using the Transwell Migration Assay. During this assay, endothelial cells are placed on the upper layer of a cell permeable membrane and a solution containing the test agent is placed below the cell permeable membrane. Following an incubation period (3-48 hours), the cells that have migrated through the membrane are stained and counted. The membrane is usually coated with some extracellular matrix component (e.g. collagen) which facilitates both adherence and migration.

In another example, microglia and/or glioblastoma cell migration and invasion can be evaluated using the Scratch Wound Healing Assay. In this assay, cells are seeded into a multiwell assay plate and allowed to attach, spread, and form a confluent monolayer. A pin tool or needle is used to scratch and remove cells from a discrete area of the confluent monolayer to form a cell-free zone into which cells at the edges of the wound can migrate. Molecules of interest as potential therapeutics are added to the wells and images of cell movement are captured at regular intervals within a 24 hour period for data analysis.

In another example, microglia and/or glioblastoma cell migration and invasion can be evaluated using the Microfluidic Chamber Assay. In this assay, the microchannels were filled with either a rat tail collagen I or Matrigel™ which was allowed to gel prior to the addition of 2,000 PC-3M cells in a 2.5 μL droplet. After an incubation period to allow the cells to adhere to the ECM, an additional 2.5 μL droplet of media containing test compounds was added and the assay device was incubated in a humidified bioassay dish for up to five days with daily exchanges of media. Cells were fixed and stained with either Alexa 594-conjugated phalloidin or Hoechst 33342 prior to imaging. Images were captured using a microscope with an automated stage and camera in conjunction with Metamorph® software for image analysis and cell counts.

In another example, microglia and/or glioblastoma cell migration and invasion can be evaluated using the Cell Exclusion Zone Assay. In this assay, a 96-well plate is populated with silicone-based cell seeding stoppers which exclude cells from attaching to a central zone. After the cells are seeded and allowed to adhere, the stoppers are removed to reveal a 2 mm diameter exclusion zone into which cells may then migrate. Stoppers remain in designated wells until assay readout to serve as pre-migration references. Cell migration data may be captured using a variety of different methods including microplate readers, microscopes, and high content imaging instruments. This stopper-based assay format allows cells to be transfected with either miRNA or siRNA reagents directly in the wells after cells are seeded. The cells are allowed to recover for 24 hours after transfection before the stoppers are removed to initiate migration of the transfected cells into the detection zone.

Another example is the brain slice assay—brain slices (250-500 uM) are prepared from mice and GFP-expressing glioma cells are implanted in them. Lateral migration of the cells on the upper side of the brain slice can be examined by fluorescent stereomicroscopic imaging to detect cells that have migrated away from the main tumor mass. This assay evaluates the invasive properties of glioma cells in under more physiologically relevant conditions.

To further illustrate this disclosure, the following examples are included. The examples should not, of course, be construed as specifically limiting the disclosure. Variations of these examples within the scope of the claims are within the purview of one skilled in the art and are considered to fall within the scope of the disclosure as described, and claimed herein. The reader will recognize that the skilled artisan, armed with the present disclosure, and skill in the art is able to prepare and use the disclosure without exhaustive examples.

### EXAMPLES

### Compound Preparation

The starting materials used in preparing the CCR1 antagonists of the disclosure are known, made by known methods, or are commercially available. It will be apparent to the skilled artisan that methods for preparing precursors and functionality related to the compounds claimed herein are generally described in the literature. The skilled artisan given the literature and this disclosure is well equipped to prepare any of the compounds.

It is recognized that the skilled artisan in the art of organic chemistry can readily carry out manipulations without further direction, that is, it is well within the scope and practice of the skilled artisan to carry out these manipulations. These include reduction of carbonyl compounds to their corresponding alcohols, oxidations, acylations, aromatic substitutions, both electrophilic and nucleophilic, etherifications, esterification and saponification and the like. These manipulations are discussed in standard texts such as March's Advanced Organic Chemistry: Reactions, Mechanisms, and Structure 7^{th} Ed., John Wiley & Sons (2013), Carey and Sundberg, Advanced Organic Chemistry 5^{th} Ed., Springer (2007), Comprehensive Organic Transformations: A Guide to Functional Group Transformations, 2^{nd} Ed., John Wiley & Sons (1999) (each incorporated herein by reference in its entirety) and the like.

The skilled artisan will readily appreciate that certain reactions are best carried out when other functionality is masked or protected in the molecule, thus avoiding any undesirable side reactions and/or increasing the yield of the reaction. Often the skilled artisan utilizes protecting groups to accomplish such increased yields or to avoid the undesired reactions. These reactions are found in the literature and are also well within the scope of the skilled artisan. Examples of many of these manipulations can be found for example in T. Greene and P. Wuts Protective Groups in Organic Synthesis, 4th Ed., John Wiley & Sons (2007), incorporated herein by reference in its entirety.

The compounds disclosed in Table 1 can be prepared using the procedures provided in the follow references. The skilled artisan is thoroughly equipped to prepare these compounds by these methods.

Compound #1 can be prepared using procedures found in Bioorganic & Medicinal Chemistry Letters (2010), 20(18), 5477-5479 and US 20090093472 (each incorporated herein by reference in its entirety) and references contained therein.

Compound #2 can be prepared using procedures found in Journal of Medicinal Chemistry (2009), 52(5), 1295-1301 and WO/2008/011392 (each incorporated herein by reference in its entirety) and references contained therein.

Compound #3 can be prepared using procedures found in WO/2004/043965 (incorporated herein by reference in its entirety) and references contained therein.

Compound #4 can be prepared using procedures found in WO/2009/011655 (incorporated herein by reference in its entirety) and references contained therein.

Compound #5 can be prepared using procedures found in Bioorganic & Medicinal Chemistry Letters (2013), 23(5), 1228-31 and WO/2005/056015 (each incorporated herein by reference in its entirety) and references contained therein.

Compound #6 can be prepared using procedures found in Journal of Medicinal Chemistry (2014), 57(18), 7550-7564 (incorporated herein by reference in its entirety) and references contained therein.

Compound #7 can be prepared using procedures found in Journal of Labelled Compounds and Radiopharmaceuticals (2006), 49(3), 253-262 and WO/2005/079769 (each incorporated herein by reference in its entirety) and references contained therein.

Compound #8 can be prepared using procedures found in Journal of Medicinal Chemistry (2005), 48(20), 6461-6471 and US 20120141471 (each incorporated herein by reference in its entirety) and references contained therein.

Compound #9 can be prepared using procedures found in Bioorganic & Medicinal Chemistry Letters (2013), 23(13), 3833-40 and Journal of Medicinal Chemistry (2014), 57(18), 7550-7564 (each incorporated herein by reference in its entirety) and references contained therein.

Compound #10 can be prepared using procedures found in US 20140057937 (incorporated herein by reference in its entirety) and references contained therein.

Compound #11 can be prepared using procedures found in WO/2012/087782 (incorporated herein by reference in its entirety) and references contained therein.

Compound #12 can be prepared using procedures found in WO/2011/137109 (incorporated herein by reference in its entirety) and references contained therein.

Compound #13 can be prepared using procedures found in WO/2011/056440 (incorporated herein by reference in its entirety) and references contained therein.

Compound #14 can be prepared using procedures found in WO/2003/105857 (incorporated herein by reference in its entirety) and references contained therein.

Compound #15 can be prepared using procedures found in Journal of Medicinal Chemistry (2001), 44(9), 1429-1435 (incorporated herein by reference in its entirety) and references contained therein.

Compound #16 can be prepared using procedures found in Bioorganic & Medicinal Chemistry (2003), 11(6), 875-884 (incorporated herein by reference in its entirety) and references contained therein.

Compound #17 can be prepared using procedures found in Bioorganic & Medicinal Chemistry Letters (2008), 18(6), 2215-2221 (incorporated herein by reference in its entirety) and references contained therein.

Compound #18 can be prepared using procedures found in Bioorganic & Medicinal Chemistry Letters (2007), 17(12), 3367-3372 (incorporated herein by reference in its entirety) and references contained therein.

Compound #19 can be prepared using procedures found in Bioorganic & Medicinal Chemistry Letters (2005), 15(23), 5160-5164 and WO/2004/037796 (each incorporated herein by reference in its entirety) and references contained therein.

Compound #20 can be prepared using procedures found in Bioorganic & Medicinal Chemistry Letters (2005), 15(23), 5160-5164 and WO/2005/103054 (each incorporated herein by reference in its entirety) and references contained therein.

Compound #21 can be prepared using procedures found in WO/2009/137338 (incorporated herein by reference in its entirety) and references contained therein.

Compound #22 can be prepared using procedures found in US 20050070549 (incorporated herein by reference in its entirety) and references contained therein.

Compound #23 can be prepared using procedures found in Organic Process Research & Development (2005), 9(4), 466-471 and US 20040087571 (incorporated herein by reference in its entirety) and references contained therein.

Compound #24 can be prepared using procedures found in Organic Process Research & Development (2010), 14(1), 72-84 (incorporated herein by reference in its entirety) and references contained therein.

### Example 2

Microglial stimulation of glioma invasion. CCR1 antagonists were screened using the glioblastoma cells and microglia invasion assay described below.

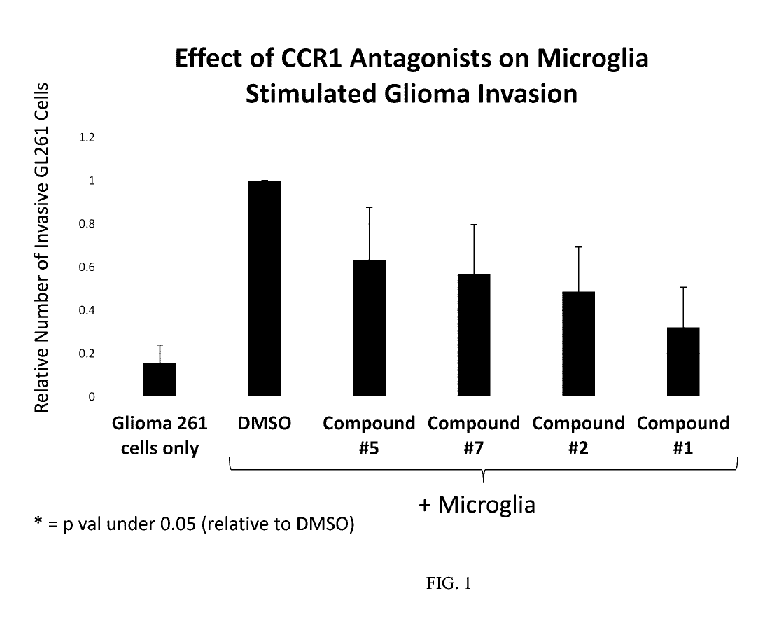

GL261 murine glioma cells expressing the fluorescent protein m-Cherry and microglial (MG) cells stained with cell tracker green (CMFDA; Invitrogen) were cocultured on Matrigel-coated invasion chambers (Becton, Dickinson and Company). Cells were plated at a density per invasion chamber of 100,000 labeled GL261 cells with an additional 150,000 unlabelled GL261 cells or with 50,000 unlabelled GL261 and 50,000 MG cells in M-SFM with 0.3% BSA. Invasion chambers were incubated for 48 hours in the presence of DMSO, or 250 nM of the CCR1 antagonist, after which they were fixed in 3.7% paraformaldehyde in PBS. Imaging of the cells on the bottom of the filter was performed using an AMG EVOS epifluorescent microscope. The extent of invasion was quantified by counting the number of glioblastoma cells (green) that were on the underside of the filter in at least seven 10× fields. The final number of invasive cells in the control was multiplied by 0.75 to normalize. FIG. 1 shows the activity of compounds #1, #2, #7, and #5.

### Example 3

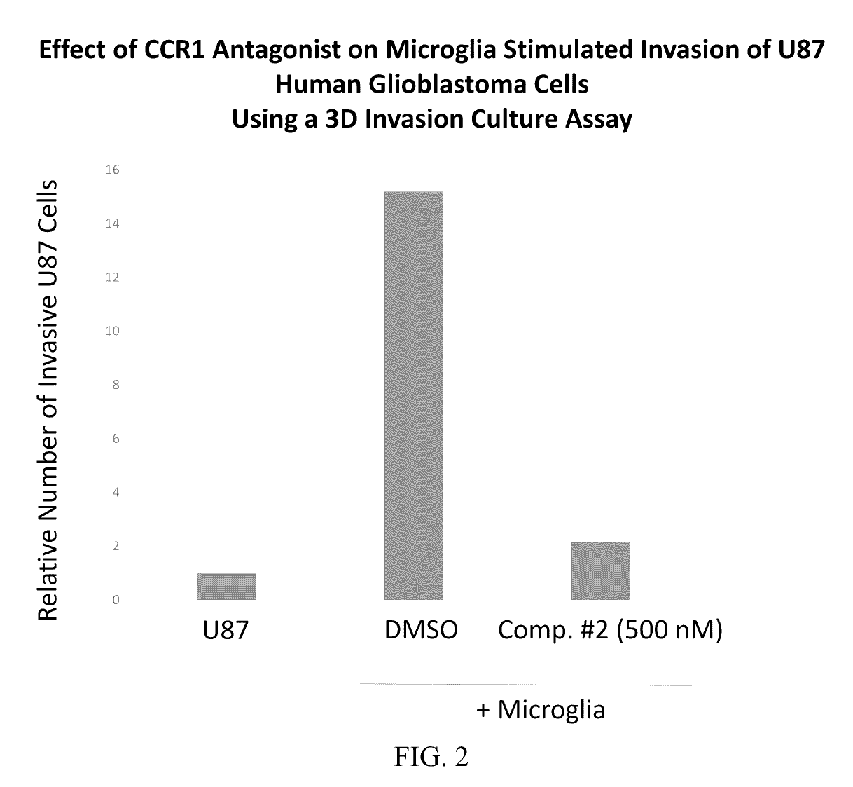

A study was prepared to determine the effect of a CCR1 antagonist described herein (i.e., compound #2 at 500 nM) on microglia stimulated invasion of U87 human glioblastoma cells using a 3D invasion culture assay. FIG. 2 shows the activity of compound #2.

U87 human glioblastoma cell lines stained with cell tracker green (CMFDA; Invitrogen) were embedded in Matrigel (10 mg/ml) with murine microglia at a density of 40,000 cells per 40 ul volume and plated within a transwell with 8 uM size filter pores. Media with serum was plated underneath the chamber and serum free media was plated on top of the matrigel layer. Cells were quantified by counting the number of U87 cells which crossed the filter after 48 hours in a tissue culture incubator. Data shown in FIG. 2 is representative of three experiments.

As shown in FIG. 2, the CCR1 antagonist compound #2 inhibited microglia sitmulated invasion of glioblastoma cells as compared to control (DMSO).

### Example 4

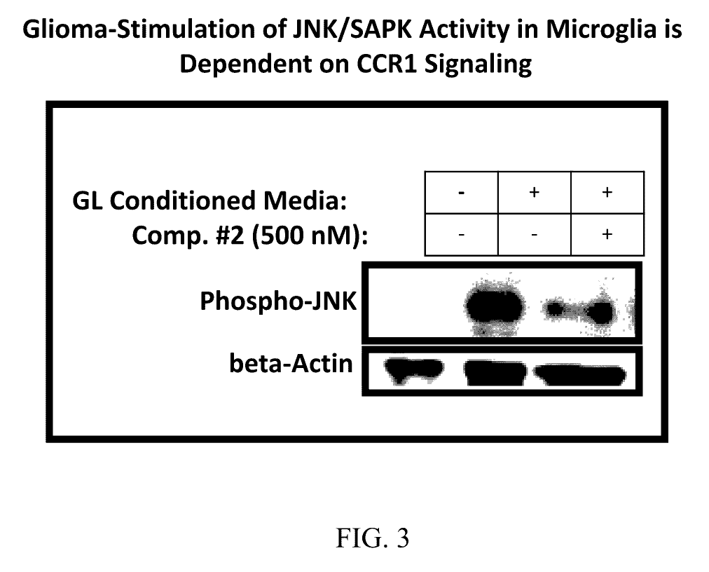

A study was prepared to determine whether glioma-stimulation of JNK/SAPK acivity in microglia is dependent on CCR1 signalling. Accordingly, a CCR1 antagonist (i.e., compound #2 at 500 nM) was used to inhibit CCR1 signalling. FIG. 3 shows the results this study.

Murine microglia were plated and serum starved overnight followed by treatment with GL261 glioma conditioned media for 30 minutes. Cells were then put on ice and harvested in sample buffer and assayed using SDS-PAGE using anti-phospho-JNK antibody (Fisher/Invitrogen). The experiment shown in FIG. 3 is representative of three independent experiments.

As shown in FIG. 3, use of the CCR1 antoginist, compound #2, indicates that glioma-stimulation of JNK/SAPK activity in microglia is dependent on CCR1 signalling.

What is claimed is:

1. A method for the treatment of tumors of the central nervous system comprising administering to a patient in need thereof a therapeutically effective amount of a CCR1 antagonist, or a pharmaceutically acceptable salt thereof.

2. The method of claim 1, wherein the tumor is selected from the group consisting of astrocytic tumors, oligodendroglial tumors, oligoastrocytic tumors, ependymal tumors, choroid plexus tumors, neuronal and mixed neuronal-glial tumors, pineal tumors, embryonal tumors, neuroblastic tumors, Glial tumors, tumors of cranial and paraspinal nerves, tumors of the meninges, tumors of the hematopoietic system, germ cell tumors, and tumors of the sellar region.

3. The method of claim 1, wherein the tumor is an astrocytic tumor.

4. The method of claim 3, wherein the astrocytic tumor is glioblastoma multiforme.

5. The method of claim 1, wherein the CCR1 antagonist is selected from the group consisting of: 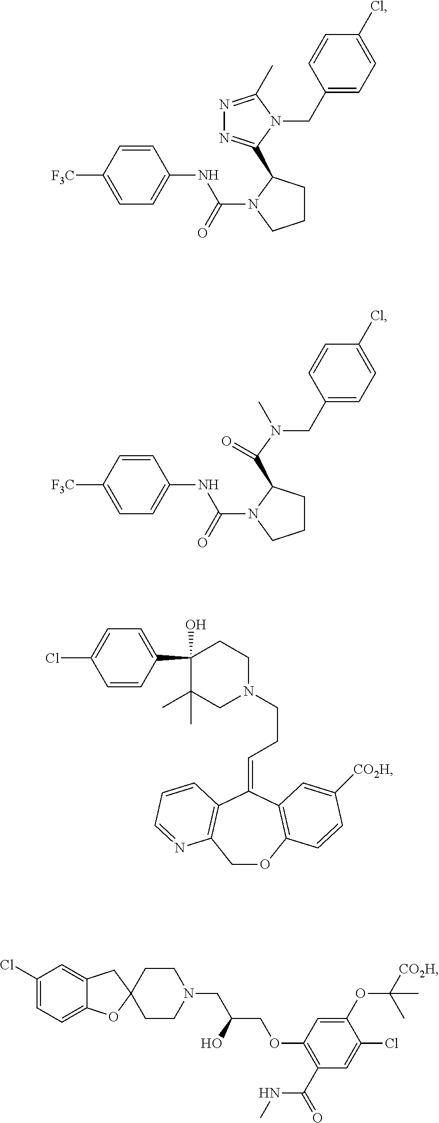 [SMILES: Cc1nnc([C@H]2CCCN2C(=O)Nc2ccc(C(F)(F)F)cc2)n1Cc1ccc(Cl)cc1 ; CN(Cc1ccc(Cl)cc1)C(=O)[C@H]1CCCN1C(=O)Nc1ccc(C(F)(F)F)cc1 ; CC1(C)CN(CC/C=C2\c3cc(C(=O)O)ccc3OCc3ncccc32)CC[C@]1(O)c1ccc(Cl)cc1 ; CNC(=O)c1cc(Cl)c(OC(C)(C)C(=O)O)cc1OC[C@@H](O)CN1CCC2(CC1)Cc1cc(Cl)ccc1O2] 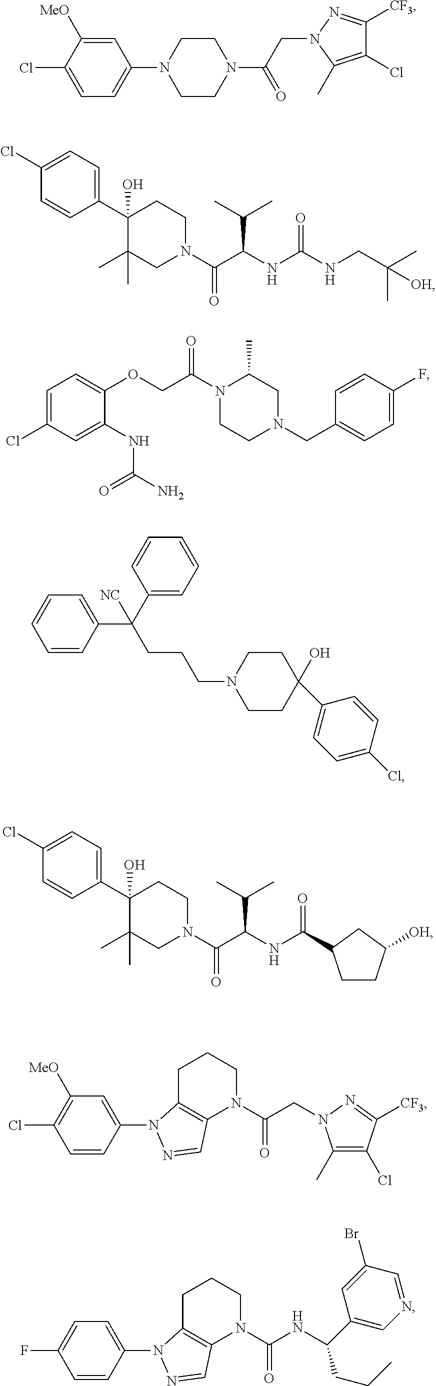 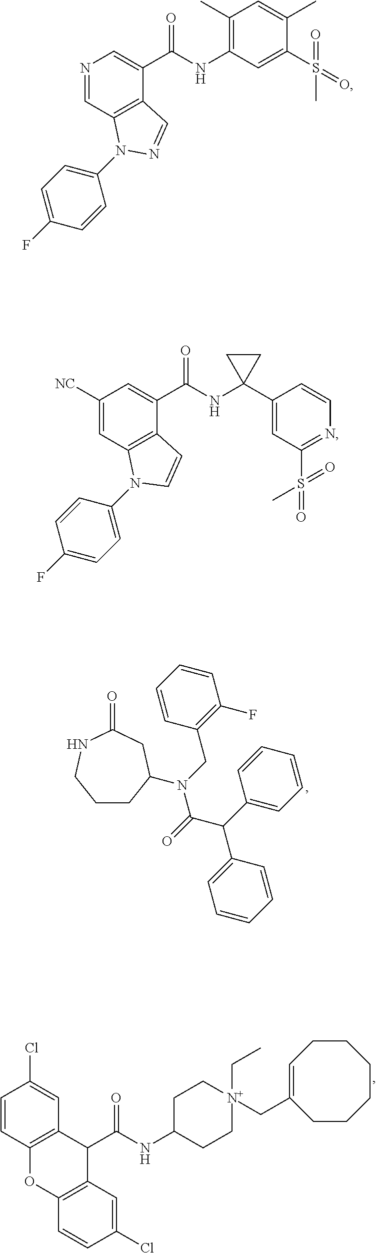 [SMILES: Cc1cc(C)c(S(C)(=O)=O)cc1NC(=O)c1cncc2c1cnn2-c1ccc(F)cc1 ; CS(=O)(=O)c1cc(C2(NC(=O)c3cc(C#N)cc4c3ccn4-c3ccc(F)cc3)CC2)ccn1 ; O=C1CC(N(Cc2ccccc2F)C(=O)C(c2ccccc2)c2ccccc2)CCCN1 ; CC[N+]1(C/C2=C/CCCCCC2)CCC(NC(=O)C2c3cc(Cl)ccc3Oc3ccc(Cl)cc32)CC1] 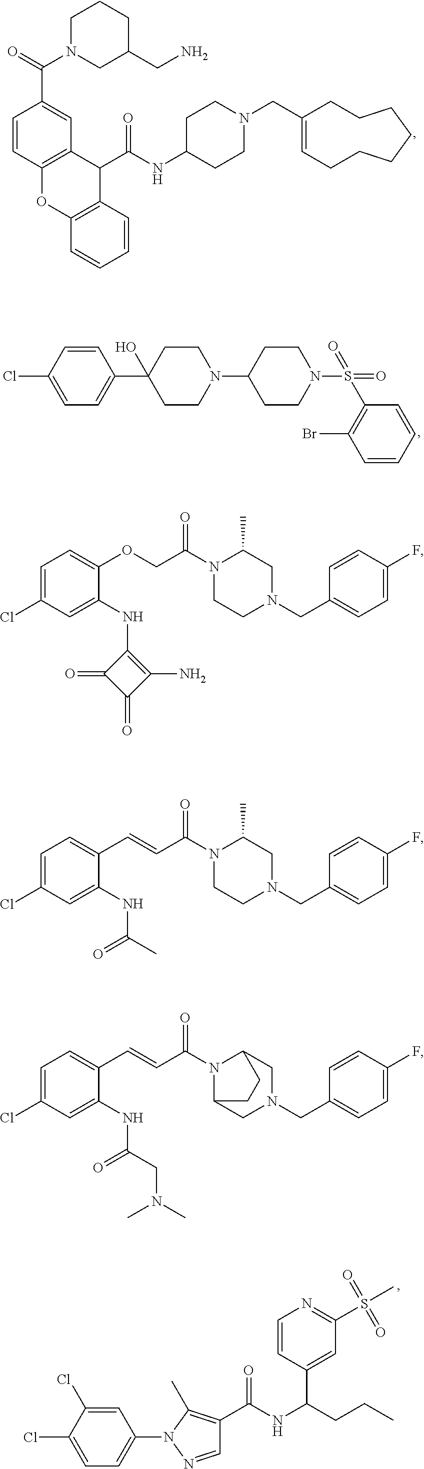 [SMILES: NCC1CCCN(C(=O)c2ccc3c(c2)C(C(=O)NC2CCN(C/C4=C/CCCCCCC4)CC2)c2ccccc2O3)C1 ; O=S(=O)(c1ccccc1Br)N1CCC(N2CCC(O)(c3ccc(Cl)cc3)CC2)CC1 ; C[C@@H]1CN(Cc2ccc(F)cc2)CCN1C(=O)COc1ccc(Cl)cc1Nc1c(N)c(=O)c1=O ; CC(=O)Nc1cc(Cl)ccc1/C=C/C(=O)N1CCN(Cc2ccc(F)cc2)C[C@H]1C ; CN(C)CC(=O)Nc1cc(Cl)ccc1/C=C/C(=O)N1C2CCC1CN(Cc1ccc(F)cc1)C2 ; CCCC(NC(=O)c1cnn(-c2ccc(Cl)c(Cl)c2)c1C)c1ccnc(S(C)(=O)=O)c1] 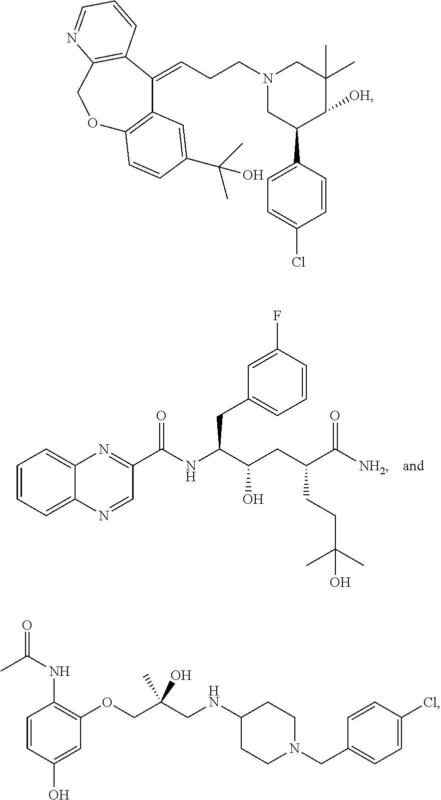
or a pharmaceutically acceptable salt thereof.

6. The method of claim 1, wherein the CCR1 antagonist is selected from the group consisting of: 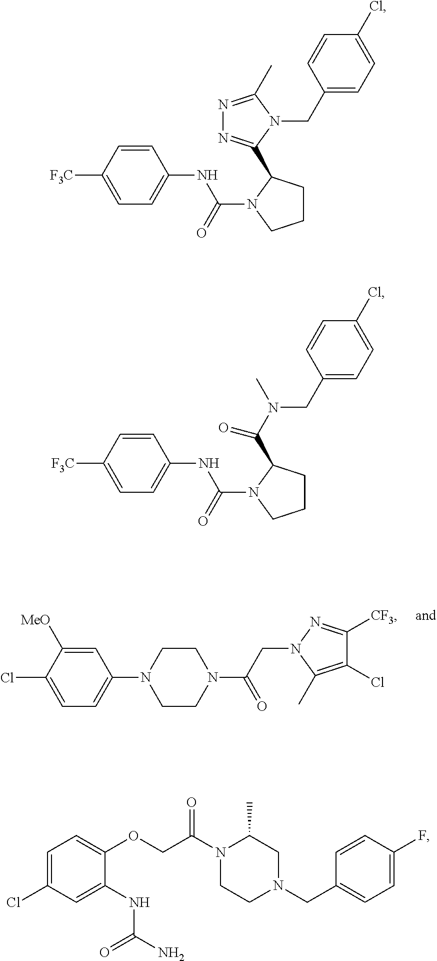 [SMILES: Cc1nnc([C@H]2CCCN2C(=O)Nc2ccc(C(F)(F)F)cc2)n1Cc1ccc(Cl)cc1 ; CN(Cc1ccc(Cl)cc1)C(=O)[C@H]1CCCN1C(=O)Nc1ccc(C(F)(F)F)cc1 ; COc1cc(N2CCN(C(=O)Cn3nc(C(F)(F)F)c(Cl)c3C)CC2)ccc1Cl ; "and" ; C[C@@H]1CN(Cc2ccc(F)cc2)CCN1C(=O)COc1ccc(Cl)cc1NC(N)=O]
or a pharmaceutically accepable salt thereof.

7. The method of claim 1, wherein the CCR1 antagonist is selected from the group consisting of: 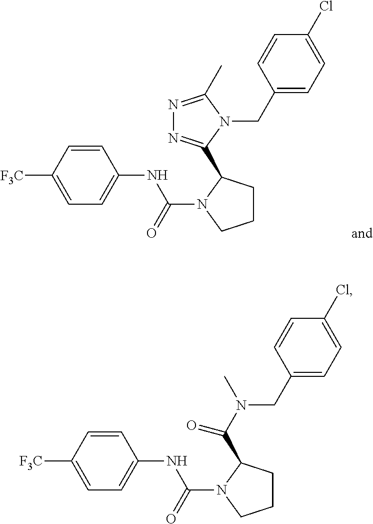 [SMILES: Cc1nnc([C@H]2CCCN2C(=O)Nc2ccc(C(F)(F)F)cc2)n1Cc1ccc(Cl)cc1 ; "and" ; CN(Cc1ccc(Cl)cc1)C(=O)[C@H]1CCCN1C(=O)Nc1ccc(C(F)(F)F)cc1]
or a pharmaceutically acceptable salt thereof.

8. The method of claim 1, wherein the CCR1 antagonist is 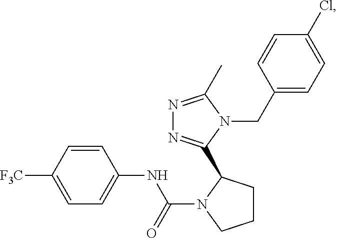 [SMILES: Cc1nnc([C@H]2CCCN2C(=O)Nc2ccc(C(F)(F)F)cc2)n1Cc1ccc(Cl)cc1]
or a pharmaceutically acceptable salt thereof.

9. The method of claim 1, comprising administering to the patient in need thereof a therapeutically effect amount of an additional active agent selected from the group consisting of Temozolomide, Bevacizumab, Carmustine, Lomustine, Semustine, cisplatin, carboplatin, vincristine, cyclophosphamide, and a combination thereof.

10. The method of claim 1, wherein administering to the patient in need thereof of the therapeutically effective amount of the CCR1 antagonist, or the pharmaceutically acceptable salt thereof, comprises a mode of administration selected from the group consisting of, intracerebral administration, intracerebroventricularl administration, oral administration, subcutaneous administration, intravenous administration, intranasal administration, topical administration, transdermal administration, intraperitoneal administration, intramuscular administration, intrapulmonary administration, vaginal administration, rectal administration, otological administration, neuro-otological administration, intraocular administration, subconjuctival administration, administration via anterior eye chamber injection, intravitreal administration, intrathecal administration, intracystical administration, intrapleural administration, administration via wound irrigation, intrabuccal administration, intra-abdominal administration, intra-articular administration, intra-aural administration, intrabronchial administration, intracapsular administration, intrameningeal administration, admininstration via inhalation, administration via endotracheal or endobronchial instillation, administration via direct instillation into pulmonary cavities, intraspinal administration, intrasynovial administration, intrathoracical administration, administration via thoracostomy irrigation, epidural administration, intratympanical administration, intracisternal administration, intravascular administration, intraventricular administration, intraosseous administration, administration via irrigation of infected bone, and administration via application as part of any admixture with a prosthetic device.

11. The method of claim 1, wherein adminstering to the patient in need thereof of the therapeutically effective amount of the CCR1 antagonist, or the pharmaceutically acceptable salt thereof, comprises a mode of administration selected from the group consisting of intranasal administration, intravenous administration, and intrathecal administration.

12. A pharmaceutical composition for treating tumors of the central nervous system, the pharmaceutical composition comprising a therapeutically effective amount of a CCR1 antagonist, or a pharmaceutically acceptable salt thereof, and a pharmaceutically acceptable carrier.

13. The pharmaceutical composition of claim 12, wherein the CCR1 antagonist is selected from the group consisting of: 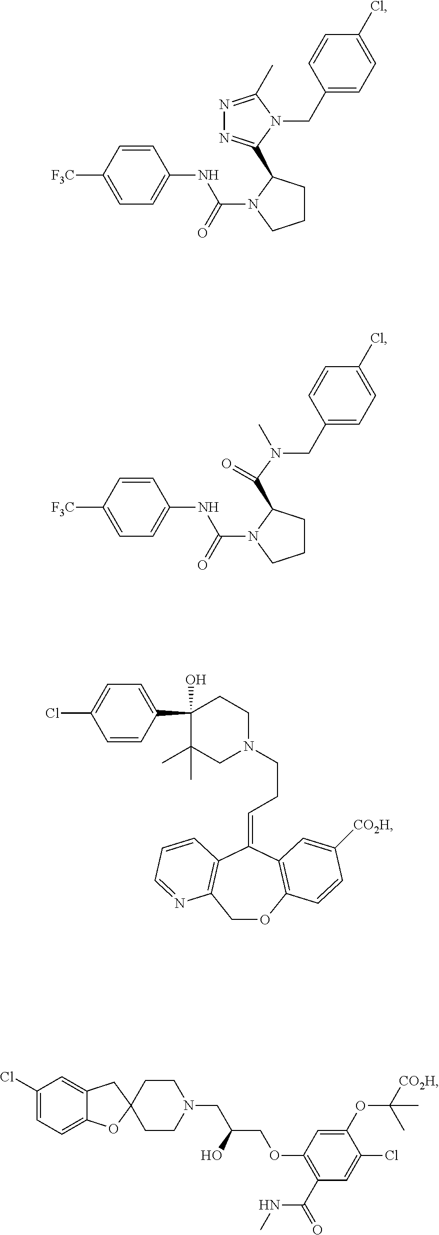 [SMILES: Cc1nnc([C@H]2CCCN2C(=O)Nc2ccc(C(F)(F)F)cc2)n1Cc1ccc(Cl)cc1 ; CN(Cc1ccc(Cl)cc1)C(=O)[C@H]1CCCN1C(=O)Nc1ccc(C(F)(F)F)cc1 ; CC1(C)CN(CC/C=C2\c3cc(C(=O)O)ccc3OCc3ncccc32)CC[C@]1(O)c1ccc(Cl)cc1 ; CNC(=O)c1cc(Cl)c(OC(C)(C)C(=O)O)cc1OC[C@@H](O)CN1CCC2(CC1)Cc1cc(Cl)ccc1O2] 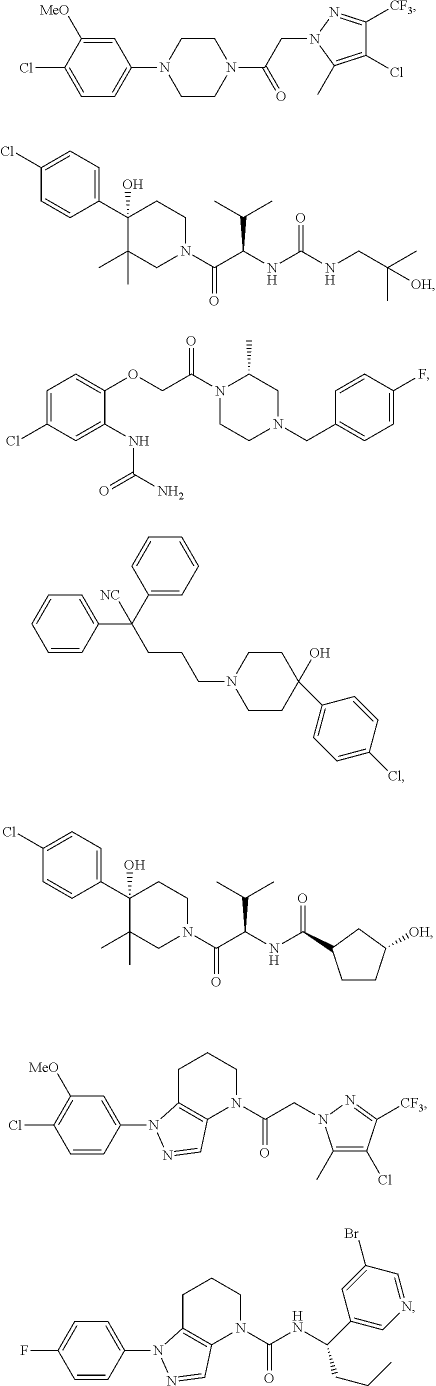 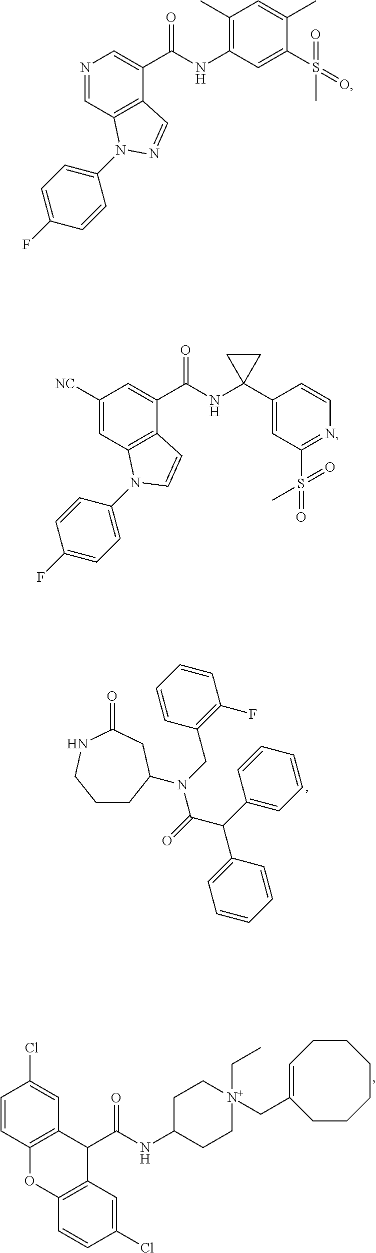 [SMILES: Cc1cc(C)c(S(C)(=O)=O)cc1NC(=O)c1cncc2c1cnn2-c1ccc(F)cc1 ; CS(=O)(=O)c1cc(C2(NC(=O)c3cc(C#N)cc4c3ccn4-c3ccc(F)cc3)CC2)ccn1 ; O=C1CC(N(Cc2ccccc2F)C(=O)C(c2ccccc2)c2ccccc2)CCCN1 ; CC[N+]1(C/C2=C/CCCCCC2)CCC(NC(=O)C2c3cc(Cl)ccc3Oc3ccc(Cl)cc32)CC1] 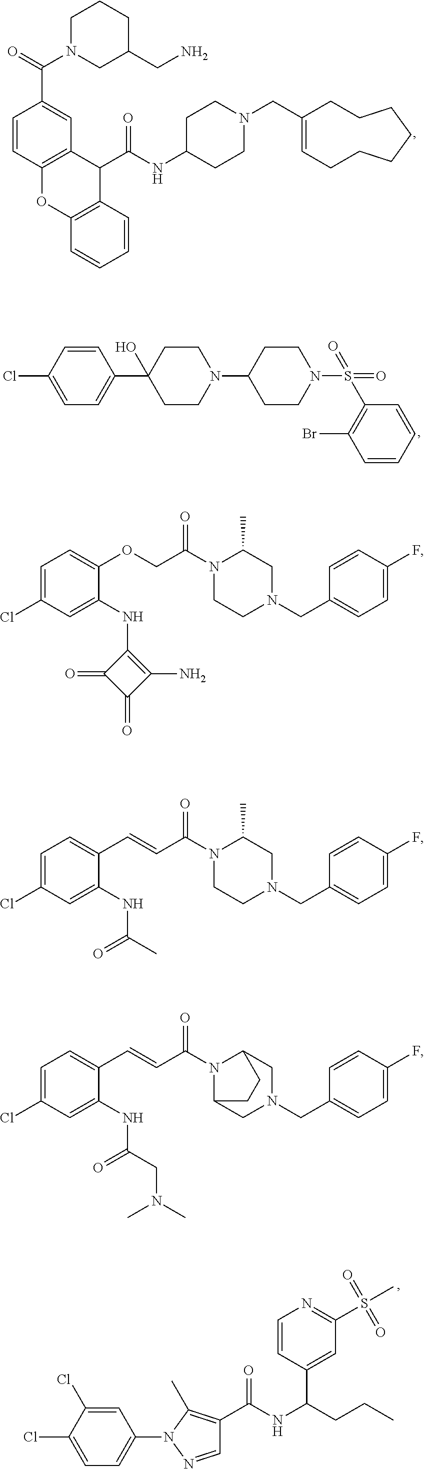 [SMILES: NCC1CCCN(C(=O)c2ccc3c(c2)C(C(=O)NC2CCN(C/C4=C/CCCCCCC4)CC2)c2ccccc2O3)C1 ; O=S(=O)(c1ccccc1Br)N1CCC(N2CCC(O)(c3ccc(Cl)cc3)CC2)CC1 ; C[C@@H]1CN(Cc2ccc(F)cc2)CCN1C(=O)COc1ccc(Cl)cc1Nc1c(N)c(=O)c1=O ; CC(=O)Nc1cc(Cl)ccc1/C=C/C(=O)N1CCN(Cc2ccc(F)cc2)C[C@H]1C ; CN(C)CC(=O)Nc1cc(Cl)ccc1/C=C/C(=O)N1C2CCC1CN(Cc1ccc(F)cc1)C2 ; CCCC(NC(=O)c1cnn(-c2ccc(Cl)c(Cl)c2)c1C)c1ccnc(S(C)(=O)=O)c1] 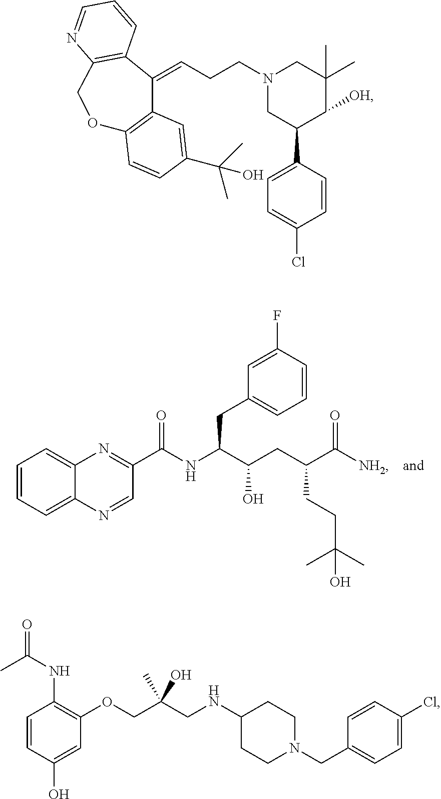
or a pharmaceutically acceptable salt thereof.

14. The pharmaceutical composition of claim 13, wherein the CCR1 antagonist is selected from the group consisting of: 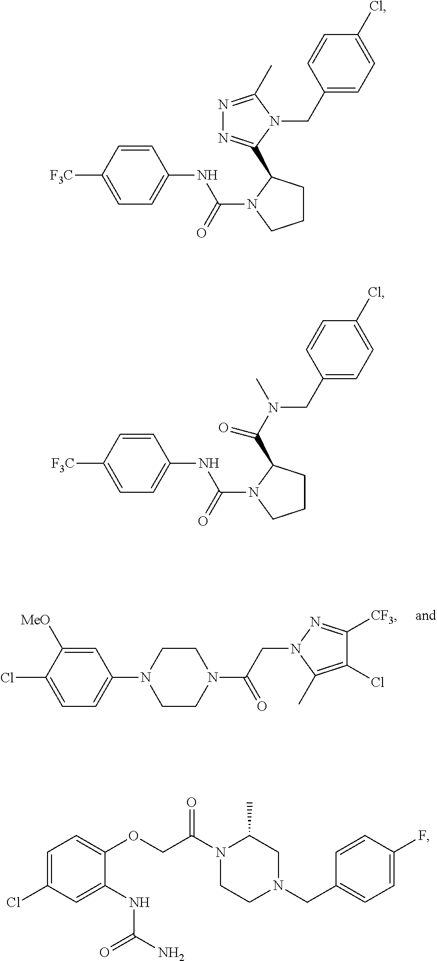 [SMILES: Cc1nnc([C@H]2CCCN2C(=O)Nc2ccc(C(F)(F)F)cc2)n1Cc1ccc(Cl)cc1 ; CN(Cc1ccc(Cl)cc1)C(=O)[C@H]1CCCN1C(=O)Nc1ccc(C(F)(F)F)cc1 ; COc1cc(N2CCN(C(=O)Cn3nc(C(F)(F)F)c(Cl)c3C)CC2)ccc1Cl ; "and" ; C[C@@H]1CN(Cc2ccc(F)cc2)CCN1C(=O)COc1ccc(Cl)cc1NC(N)=O]
or a pharmaceutically accepable salt thereof.

15. The pharmaceutical composition of claim 14, wherein the CCR1 antagonist is selected from the group consisting of: 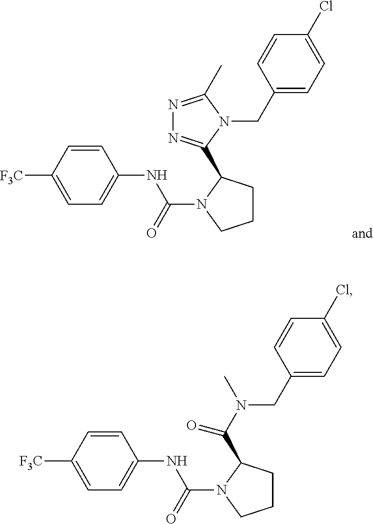 [SMILES: Cc1nnc([C@H]2CCCN2C(=O)Nc2ccc(C(F)(F)F)cc2)n1Cc1ccc(Cl)cc1 ; "and" ; CN(Cc1ccc(Cl)cc1)C(=O)[C@H]1CCCN1C(=O)Nc1ccc(C(F)(F)F)cc1]
or a pharmaceutically acceptable salt thereof.

16. The pharmaceutical composition of claim 15, wherein the CCR1 antagonist is 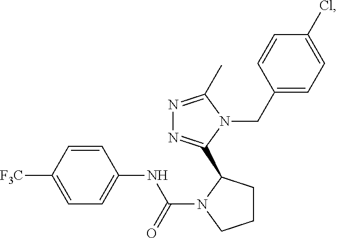 [SMILES: Cc1nnc([C@H]2CCCN2C(=O)Nc2ccc(C(F)(F)F)cc2)n1Cc1ccc(Cl)cc1]
or a pharmaceutically acceptable salt thereof.

17. The pharmaceutical composition of claim 12, comprising a therapeutically effective amount of an additional active agent selected from the group consisting of Temozolomide, Bevacizumab, Carmustine, Lomustine, Semustine, cisplatin, carboplatin, vincristine, cyclophosphamide, and a combination thereof.

18. The pharmaceutical composition of claim 17, wherein the tumor is selected from the group consisting of astrocytic tumors, oligodendroglial tumors, oligoastrocytic tumors, ependymal tumors, choroid plexus tumors, neuronal and mixed neuronal-glial tumors, pineal tumors, embryonal tumors, neuroblastic tumors, Glial tumors, tumors of cranial and paraspinal nerves, tumors of the meninges, tumors of the hematopoietic system, germ cell tumors, and tumors of the sellar region.

19. The pharmaceutical composition of claim 18, wherein the tumor is an astrocytic tumor.

20. The pharmaceutical composition of claim 19, wherein the astrocytic tumor is glioblastoma multiforme.

21. A method of treating a tumor by inhibiting tumor-associated macrophage invasion in a patient in need thereof, the method comprising administering to the patient a therapeutically effective amount of a CCR1 antagonist, or a pharmaceutically acceptable salt thereof.

22. The method of claim 21, wherein the tumor is selected from the group consisting of astrocytic tumors, oligodendroglial tumors, oligoastrocytic tumors, ependymal tumors, choroid plexus tumors, neuronal and mixed neuronal-glial tumors, pineal tumors, embryonal tumors, neuroblastic tumors, Glial tumors, tumors of cranial and paraspinal nerves, tumors of the meninges, tumors of the hematopoietic system, germ cell tumors, and tumors of the sellar region.

23. The method of claim 21, wherein wherein the CCR1 antagonist is selected from the group consisting of: 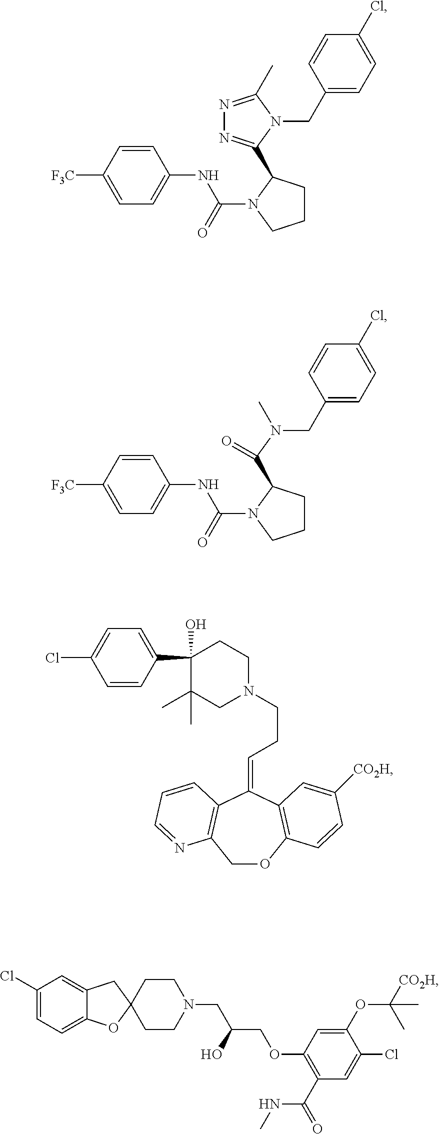 [SMILES: Cc1nnc([C@H]2CCCN2C(=O)Nc2ccc(C(F)(F)F)cc2)n1Cc1ccc(Cl)cc1 ; CN(Cc1ccc(Cl)cc1)C(=O)[C@H]1CCCN1C(=O)Nc1ccc(C(F)(F)F)cc1 ; CC1(C)CN(CC/C=C2\c3cc(C(=O)O)ccc3OCc3ncccc32)CC[C@]1(O)c1ccc(Cl)cc1 ; CNC(=O)c1cc(Cl)c(OC(C)(C)C(=O)O)cc1OC[C@@H](O)CN1CCC2(CC1)Cc1cc(Cl)ccc1O2] 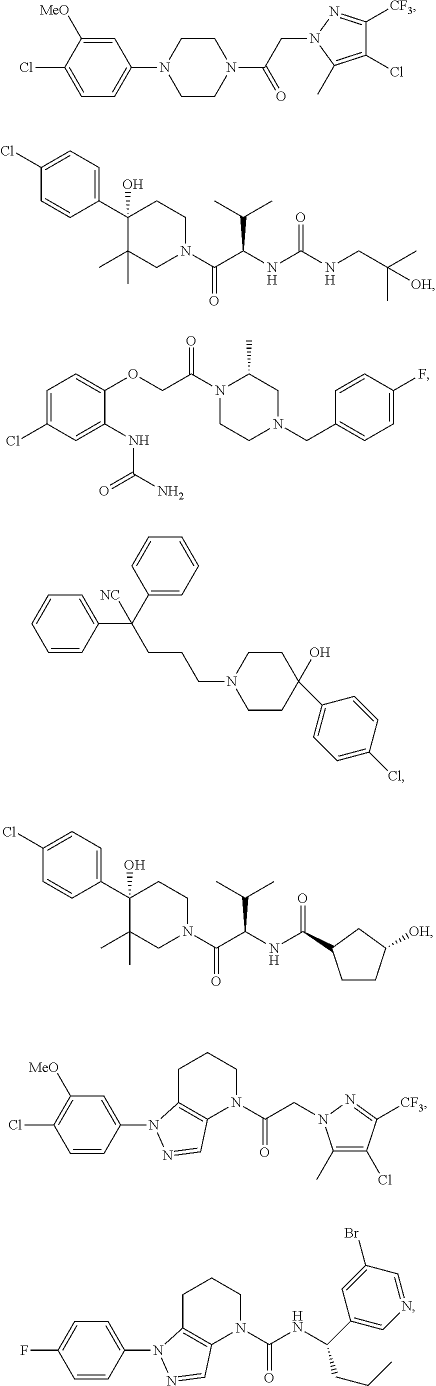 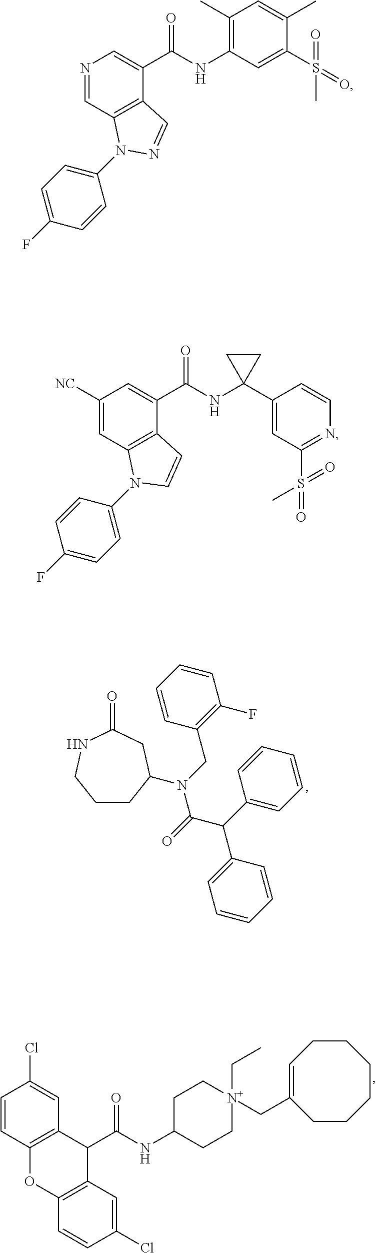 [SMILES: Cc1cc(C)c(S(C)(=O)=O)cc1NC(=O)c1cncc2c1cnn2-c1ccc(F)cc1 ; CS(=O)(=O)c1cc(C2(NC(=O)c3cc(C#N)cc4c3ccn4-c3ccc(F)cc3)CC2)ccn1 ; O=C1CC(N(Cc2ccccc2F)C(=O)C(c2ccccc2)c2ccccc2)CCCN1 ; CC[N+]1(C/C2=C/CCCCCC2)CCC(NC(=O)C2c3cc(Cl)ccc3Oc3ccc(Cl)cc32)CC1] 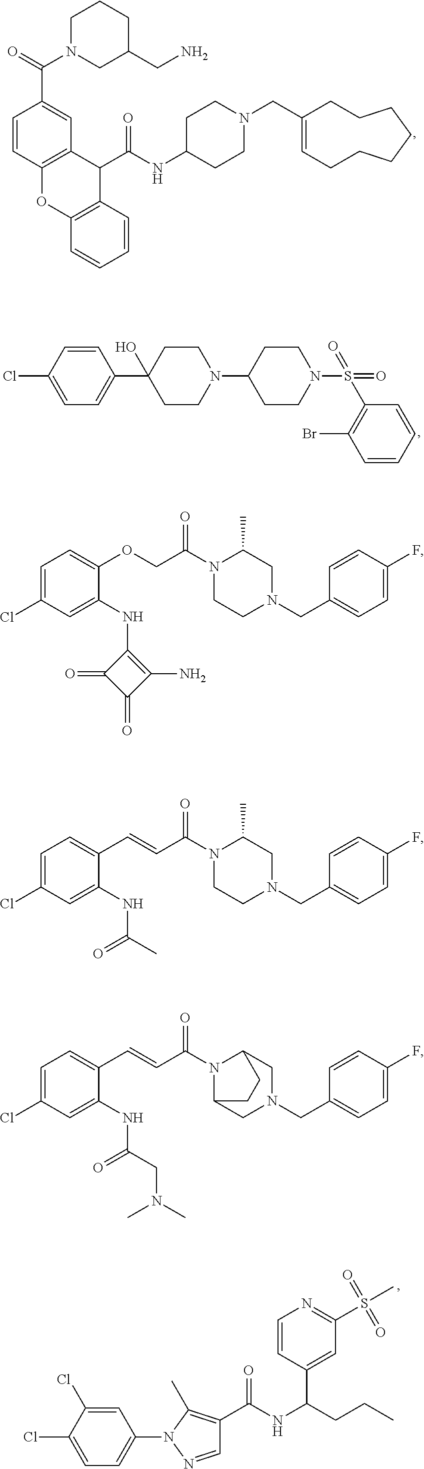 [SMILES: NCC1CCCN(C(=O)c2ccc3c(c2)C(C(=O)NC2CCN(C/C4=C/CCCCCCC4)CC2)c2ccccc2O3)C1 ; O=S(=O)(c1ccccc1Br)N1CCC(N2CCC(O)(c3ccc(Cl)cc3)CC2)CC1 ; C[C@@H]1CN(Cc2ccc(F)cc2)CCN1C(=O)COc1ccc(Cl)cc1Nc1c(N)c(=O)c1=O ; CC(=O)Nc1cc(Cl)ccc1/C=C/C(=O)N1CCN(Cc2ccc(F)cc2)C[C@H]1C ; CN(C)CC(=O)Nc1cc(Cl)ccc1/C=C/C(=O)N1C2CCC1CN(Cc1ccc(F)cc1)C2 ; CCCC(NC(=O)c1cnn(-c2ccc(Cl)c(Cl)c2)c1C)c1ccnc(S(C)(=O)=O)c1] 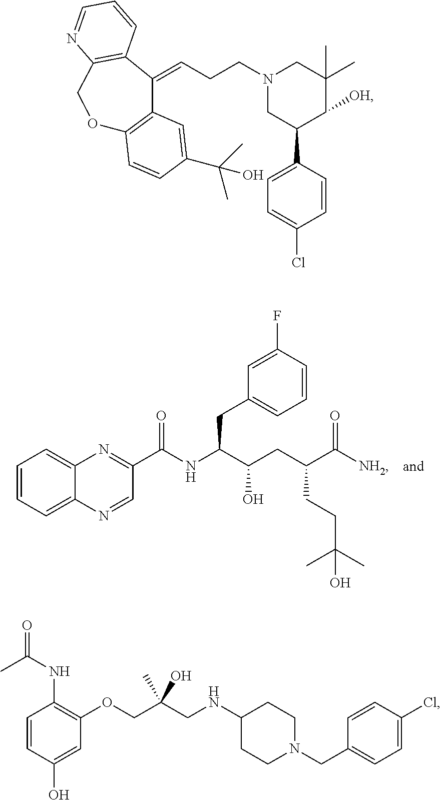
or a pharmaceutically acceptable salt thereof.
